(19) United States
(12) Patent Application Publication
(10) Pub. No.: US 2026/0053891 A1
(43) Pub. Date: Feb. 26, 2026

(54) METHODS AND COMPOSITIONS FOR TREATING CANCER

(71) Applicant: THE REGENTS OF THE UNIVERSITY OF MICHIGAN, Ann Arbor, MI (US)

(72) Inventors: Peng Zhang, Ann Arbor, MI (US); Zhimin Chen, Ann Arbor, MI (US); Jiandie Lin, Ann Arbor, MI (US)

(21) Appl. No.: 19/104,085

(22) PCT Filed: Aug. 15, 2023
(86) PCT No.: PCT/US2023/072235

§ 371 Date: Feb. 14, 2025

Related U.S. Application Data
(60) Provisional application No. 63/371,426, filed on Aug. 15, 2022.

(51) Int. Cl. A61K 38/18; A61K 45/06; A61K 47/54; A61K 47/68; A61P 35/00

(52) U.S. Cl. CPC A61K 38/1883; A61K 45/06; A61K 47/543; A61K 47/6811; A61P 35/00

(57) ABSTRACT

Provided herein are methods and compositions for treating cancer, particularly the methods and compositions comprising a neuregulin 4 (NRG4) polypeptide.

### CROSS-REFERENCE TO RELATED APPLICATIONS

This application claims the benefit of U.S. Provisional Application No. 63/371,426, filed Aug. 15, 2022, the content of which is herein incorporated by reference in its entirety.

### STATEMENT REGARDING FEDERALLY SPONSORED RESEARCH OR DEVELOPMENT

This invention was made with government support under DK102456 awarded by the National Institutes of Health. The government has certain rights in the invention.

### SEQUENCE LISTING STATEMENT

The contents of the electronic sequence listing titled UM-41169.601.xml (Size: 3,832 bytes; and Date of Creation: Aug. 14, 2023) is herein incorporated by reference in its entirety.

### FIELD

The present disclosure relates to methods and compositions for treating cancer, particularly the disclosure relates to methods and compositions comprising a neuregulin 4 (NRG4) polypeptide.

### BACKGROUND

Several pathogenic mechanisms have been implicated in the development of hepatic steatosis, including insulin resistance, adipose tissue dysfunction, and dysregulations of hepatic lipid metabolism. Hepatic steatosis often exists in a clinically benign state, but frequently progresses to non-alcoholic steatohepatitis (NASH), a more severe metabolic liver disease characterized by persistent liver injury, inflammation, and fibrosis. Chronic liver injury in NASH increases the risk for end-stage liver disease such as cirrhosis and liver cancer. Although, potential therapies targeting diverse pathways, including hepatic metabolism, inflammation, and liver fibrosis, are being evaluated, no effective pharmacological therapies targeting NASH, and its associated sequelae, are currently available.

### SUMMARY

Provided herein are methods for treating or preventing cancer. In some embodiments, the methods are for treating or preventing cancer in a subject. In some embodiments, the subject is human. In some embodiments, the methods suppress or eliminate cancer metastasis, decrease tumor growth, prevent tumor recurrences, or any combination thereof.

In some embodiments, the methods comprise administering to the subject an effective amount of a Neuregulin 4 (NRG4) polypeptide, or a nucleic acid encoding thereof.

In some embodiments, the NRG4 polypeptide comprises the amino acid sequence of SEQ ID NO: 1, an NRG4 variant comprising at least 70% identity to SEQ ID NO: 1, or a biologically active fragment thereof. In some embodiments, the NRG4 polypeptide comprises a biologically active fragment comprising amino acids 5-46, 5-55, 5-62, 1-46, 1-55, 1-52, 1-53, 4-52, 4-53, or 1-62 of SEQ ID NO: 1.

In some embodiments, the NRG4 polypeptide further comprises a lipid moiety.

In some embodiments, the NRG4 polypeptide is linked to a Fc domain (e.g., in a fusion protein). Thus, in some embodiments, the methods comprise administering to the subject an effective amount of a fusion protein comprising a Neuregulin 4 (NRG4) polypeptide and an Fc domain, or a nucleic acid encoding thereof. In some embodiments, the NRG4 polypeptide is linked to the Fc domain by a flexible linker.

In some embodiments, the Fc domain is derived from an immunoglobulin IgG Fc domain.

In some embodiments, the methods further comprise administering at least one additional therapeutic agent. In some embodiments, the at least one additional therapeutic agent is an immune checkpoint inhibitor, a receptor tyrosine kinase inhibitor, or a combination thereof.

In some embodiments, the cancer comprises a solid tumor or hematological cancer. In some embodiments, the cancer is metastatic cancer. In some embodiments, the cancer is liver cancer. In some embodiments, the liver cancer is selected from a hepatocellular carcinoma (HCC), a hepatoblastoma, a cholangiocarcinoma, a cholangiocellular cystadenocarcinoma, an angiosarcoma, a hemangioendothelioma, or a combination thereof. In some embodiments, the liver cancer is hepatocellular carcinoma (HCC). In some embodiments, the liver cancer is nonalcoholic steatohepatitis (NASH)-associated liver cancer. In some embodiments, the subject has or is suspected of having non-alcoholic steatohepatitis.

Also provided herein are compositions comprising a Neuregulin 4 (NRG4) polypeptide, or a nucleic acid encoding thereof; and one or more additional therapeutic agents selected from the group consisting of: checkpoint inhibitors, receptor tyrosine kinase inhibitors, and combinations thereof.

In some embodiments, the NRG4 polypeptide comprises the amino acid sequence of SEQ ID NO: 1, an NRG4 variant comprising at least 70% identity to SEQ ID NO: 1, or a biologically active fragment thereof. In some embodiments, the NRG4 polypeptide comprises a biologically active fragment comprising amino acids 5-46, 5-55, 5-62, 1-46, 1-55, 1-52, 1-53, 4-52, 4-53, or 1-62 of SEQ ID NO: 1.

In some embodiments, the NRG4 polypeptide is linked to a Fc domain. In some embodiments, the NRG4 polypeptide is linked to the Fc domain by a flexible linker. In some embodiments, the Fc domain is derived from an immunoglobulin IgG Fc domain.

In some embodiments, the NRG4 polypeptide further comprises a lipid moiety.

Further provided are methods of treating or preventing cancer in a subject comprising administering to the subject a composition disclosed herein. In some embodiments, the composition comprises a Neuregulin 4 (NRG4) polypeptide, or a nucleic acid encoding thereof. In some embodiments, the composition comprises a fusion protein comprising a Neuregulin 4 (NRG4) polypeptide and an Fc domain, or a nucleic acid encoding thereof. In some embodiments, the composition comprises a Neuregulin 4 (NRG4) polypeptide, or a nucleic acid encoding thereof, and one or more additional therapeutic agents selected from checkpoint inhibitors, receptor tyrosine kinase inhibitors, or combinations thereof. In some embodiments, the composition comprises a fusion protein comprising a Neuregulin 4 (NRG4) polypeptide and an Fc domain, or a nucleic acid encoding thereof, and one or more additional therapeutic agents selected from checkpoint inhibitors, receptor tyrosine kinase inhibitors, or combinations thereof.

In some embodiments, the cancer comprises a solid tumor or hematological cancer. In some embodiments, the cancer is metastatic cancer.

In some embodiments, the cancer is liver cancer. In some embodiments, the liver cancer is selected from: a hepatocellular carcinoma (HCC), a hepatoblastoma, a cholangiocarcinoma, a cholangiocellular cystadenocarcinoma, an angiosarcoma, a hemangioendothelioma, and combinations thereof. In some embodiments, the liver cancer is hepatocellular carcinoma (HCC). In some embodiments, the liver cancer is nonalcoholic steatohepatitis (NASH)-associated liver cancer. In some embodiments, the subject has or is suspected of having non-alcoholic steatohepatitis.

Other aspects and embodiments of the disclosure will be apparent in light of the following detailed description.

### BRIEF DESCRIPTION OF THE DRAWINGS

FIGS. 1A-1J show the induction and regulation of TAM-like microphages in NASH liver. FIG. 1A is a heatmap of top 20 marker genes for the macrophage subclusters. FIG. 1B is feature plots illustrating macrophage gene expression. FIG. 1C is virtual flow analysis of intrahepatic macrophages by gating for Csf1r and Apoe, Trem2, or Tgfbr1 mRNA expression. FIG. 1D is fluorescence images of liver sections from Trem2-Cre/Rosa26-tdTomato mice under FIG. 1E is RNA velocity analysis of macrophage gene expression. Arrows denote likely trajectory of cell states among different subpopulations. FIG. 1F is flow cytometry analysis of liver macrophages in mice fed NASH diet following bone marrow transplantation. FIG. 1G is a bubble plot illustrating relative mRNA expression for genes encoding secreted factors (top) and membrane proteins (bottom) with enriched expression in NAMs. FIG. 1H is violin plots of gene expression among different macrophage subclusters. FIG. 1I qPCR analysis of Tgfb expression in chow and NASH liver. FIG. 1J is qPCR analysis of gene expression in cultured BMDMs treated with vehicle or 2.5 ng/ml TGFβ for 24 hrs. Data in FIGS. 1F, 1I, and 1J represent mean±SEM; two-tailed unpaired Student's t-test. **p<0.01, ***p<0.001, ****p<0.0001.

FIGS. 2A-2J show that NASH pathogenesis triggers CD8+ T cell exhaustion in the liver. FIG. 2A is uniform manifold approximation and projection (UMAP) representation illustrating T cells among liver NPCs (top) and three T cell subclusters (bottom). FIG. 2B is a volcano plot of gene expression using averaged values of normalized expression levels for CD8+ T cells from chow and NASH livers. X-axis indicates log-transformed fold change of gene expression between NASH and chow livers. FIG. 2C is a heatmap of a subset of genes differentially expressed in CD8+ T cells from chow and NASH mouse livers. FIG. 2D is a dot plot illustrating relative abundance of CD8+ T cells expressing Cd8a in combination with the indicated genes in chow and NASH livers. FIG. 2E is qPCR analysis of hepatic gene expression in mice fed chow (n=7) or NASH (n=7) diet for 4 months. FIG. 2F is confocal images of anti-PD1 immunofluorescence staining on liver sections. Scale bars=100 μm. FIG. 2G is flow cytometry analysis of PD1 expression in intrahepatic T cells from mice fed chow (n=5) and NASH (n=5) diet. FIG. 2H is flow cytometry analysis of intracellular IFNγ and IL-2. Liver NPCs from mice fed chow (n=8) or NASH (n=6) diet for 5 months were treated with PMA/ionomycin for 6 hrs. FIG. 2I is CFSE proliferation assay of CD8+ T cells. NPCs from chow (n=3) or NASH (n=3) mouse livers were incubated with CD3/CD28 Dynabead for 24 and 120 hrs. FIG. 2J is qPCR analysis of gene expression in liver biopsies from non-NASH (n=7) and NASH patients (n=7). Data in FIGS. 2E, and 2G-2J represent mean±SEM; two-tailed unpaired Student's ttest. *p<0.05, **p<0.01, ***p<0.001, ****p<0.0001.

FIGS. 3A-3H show that NRG4 serves as a hormonal checkpoint for NASH-associated HCC. FIG. 3A is a volcano plot illustrating differential gene expression. Bulk liver RNA sequencing was performed on three pairs of pooled NASH diet-fed WT and NRG4 KO mice. X-axis indicates log-transformed fold change of gene expression between KO and WT mice. FIG. 3B is gene ontology analysis of upregulated (red, lower) and downregulated (blue, top) genes in FIG. 3A. FIG. 3C is qPCR analysis of hepatic gene expression in WT (n=10) and NRG4 KO (n=11) mice fed NASH diet for 6 months. FIG. 3D is a schematic outline of DEN/NASH liver tumor study using male WT (n=22) and NRG4 KO (n=28) mice. NASH diet feeding was initiated at 5 weeks of age. FIG. 3E is a metabolic parameters and tumor count in WT (n=22) and NRG4 KO (n=28) mice subjected to the DEN/NASH protocol. FIG. 3F is a schematic outline of DEN/NASH liver tumor study using male WT (n=22) and NRG4 KO (n=28) mice. NASH diet feeding was initiated at 5 weeks of age. FIG. 3G is metabolic parameters and tumor count in the treated mice. FIG. 3H is liver appearance and histology. Scale bar=200 μm. Data in FIGS. 3C-3E and 3G represent mean±SEM; two-tailed unpaired Student's t-test. *p<0.05, **p<0.01, ***p<0.001, ****p<0.0001.

FIGS. 4A-4J show the effects of NRG4 deficiency on the liver immune microenvironment. Single-cell RNAseq analysis of liver NPCs isolated from WT (n=4) and Nrg4 KO (n=4) mice following 7 months of NASH diet feeding. FIG. 4A is macrophage subclusters and feature plots. FIG. 4B is pie chart of macrophage cell count from WT (red) and NRG4 KO (blue) livers in each subcluster. FIG. 4C is confocal images of liver immunofluorescence staining. Scale bar=100 μm. FIG. 4D is immunoblots of total liver lysates from WT and NRG4 KO mice fed NASH diet for 6 months. FIG. 4E is T cell subclusters and feature plots. FIG. 4F is virtual flow analysis of intrahepatic CD8+ T cells by gating for Cd8a in combination with Pdcd1 or Lag3 mRNA levels. FIG. 4G is confocal images of liver immunofluorescence staining. Scale bar=100 μm. FIG. 4H is flow cytometry analysis of PD1 expression in intrahepatic CD8+ T cells from NASH diet fed WT (n=8) and NRG4 KO (n=9) mice. FIG. 4I is CFSE proliferation assay of CD8+ T cells from WT (n=8) and NRG4 KO (n=10) mice fed NASH diet for 5 months. FIG. 4J is anti-PDL1 treatment study. WT and NRG4 KO mice were subjected to the DEN/NASH tumor induction protocol followed by treatments twice a week: WT IgG2b (n=10), WT anti-PDL1 (n=15), Nrg4 KO IgG2b (n=11), Nrg4 KO anti-PDL1 (n=10). Data in FIGS. 4G, 4I and 4J represent mean±SEM; two-tailed unpaired Student's t-test. *p<0.05, ** p<0.01, and ****p<0.0001.

FIGS. 5A-5H show that inhibition of tumor-prone liver microenvironment and NASH-HCC by NRG4. FIG. 5A is a schematic overview of DEN/NASH liver tumor study using male WT (n=14) and NRG4 TG (n=14) mice. NASH diet feeding was initiated at 5 weeks of age. FIG. 5B is graphs of metabolic parameters and tumor count in treated mice. FIG. 5C is images of liver appearance. FIG. 5D is qPCR analysis of gene expression WT and NRG4 TG mouse livers. FIG. 5E is a schematic diagram of hNRG4-Fc fusion protein design and study outline. FIG. 5F is H&E histology and Sirius red staining of liver sections from transduced mice. Scale bar=200 μm. FIG. 5G is liver hydroxyproline content in mice transduced with AAV-Fc (n=7) or AAV-hNRG4-Fc (n=8). FIG. 5H is qPCR analysis of hepatic gene expression in transduced mice. Data in FIGS. 5B, 5D, and 5G-5H represent mean±SEM; two-tailed unpaired Student's t-test. *p<0.05, **p<0.01, ***p<0.001.

FIGS. 6A-6F show that suppression of oncogene-induced HCC by recombinant hNRG4-Fc fusion protein. FIG. 6A is a graph of plasma concentrations of hNRG4-Fc, as measured by hIgG1 Fc ELISA, at different time points following a single injection of the fusion protein (2 mg/kg, i.p.). FIG. 6B Immunoblots of total Min6 cell lysates treated with Fc, hNRG4-Fc, or hNRG4 peptide at 0.8, 4, 20, or 100 nM for 15 minutes. FIG. 6C is a schematic outline of hNRG4-Fc fusion protein treatment study, and the metabolic parameters and tumor burden in the treated mice. Four-month-old male mice were transduced with AAV-cMYC/AAV-nRAS followed by weekly treatment with Fc (2.5 mg/kg, n=13) or hNRG4-Fc at 0.5 mg/kg (n=11) or 2.5 mg/kg (n=14). FIG. 6D is qPCR analysis of hepatic gene expression in treated mice. FIG. 6E is images of liver appearance and histology. Scale bar=200 μm. FIG. 6F is survival curves of transduced mice treated with 1.5 mg/kg of Fc (n=15) or hNRG4-Fc (n=15) fusion protein. Data in FIGS. 6C and 6D represent mean±SEM; two-tailed unpaired Student's t-test. *p<0.05, **p<0.01, ***p<0.001.

FIGS. 7A-7E show that interaction between NRG4 and the liver immune microenvironment in NASH-HCC. FIG. 7A is a heatmap illustrating expression patterns of NRG4-regulated genes among different liver cell types. FIG. 7B is hNRG4-Fc and anti-PDL1 co-treatment study. WT male mice transduced with AAV oncogenes were randomly divided into four treatment groups: IgG2b+Fc (n=15), IgG2b+hNRG4-Fc (n=15), anti-PDL1+Fc (n=15), anti-PDL1+hNRG4-Fc (n=15). FIG. 7C is tumor burden in transduced WT and Trem2 KO mice: WT Fc (n=12), Trem2KO Fc (n=15), WT hNRG4-Fc (n=16), Trem2KO hNRG4-Fc (n=11). FIG. 7D is CFSE proliferation assay of splenic CD8+ T cells from OT-1 transgenic mice cocultured with BMDMs from WT and Trem2 KO mice in the absence or presence of OVA. FIG. 7E is a model depicting NRG4 as a hormonal checkpoint in NASH-associated HCC. Data in FIGS. 7B and 7C represent mean±SEM; two-tailed unpaired Student's t-test. *p<0.05, ** p<0.01, ***p<0.001, ****p<0.0001.

FIGS. 8A-8F show the induction and regulation of TAM-like macrophages in NASH liver. FIG. 8A is UMAP representation of 11 major liver cell clusters. FIG. 8B is feature plots illustrating classical and non-classical macrophage marker genes expression. FIG. 8C is feature plots illustrating cell distribution between Chow and NASH diet treatment. FIG. 8D is graphs of cell count (left) and percentage of total macrophages (right) from chow (blue) and NASH (red) livers for macrophage subclusters. FIG. 8E is qPCR analysis of hepatic gene expression in control mice (n=5) and DEN-treated mice fed NASH diet for 4 (n=5) or 6 (n=7) months. Data represent mean +SEM; two-tailed unpaired Student's t-test. *p<0.05, ** p<0.01, ***p<0.001, ****p<0.0001. FIG. 8F is UMI expression of Tgfb1-3 in different cell types of liver.

FIGS. 9A-9G show single-cell analysis of intrahepatic T cells. FIG. 9A is cell count from chow (blue) and NASH (red) livers in each T cell subclusters. FIG. 9B is a volcano plot illustrating differential gene expression for T cells. FIG. 9C is pathway analysis of differentially expressed genes in CD8+ T cells. FIG. 9D is a heat map of a subset of genes differentially expressed in T cells from chow and NASH livers. FIG. 9E is CD8+ T cell subclusters and cell count. FIG. 9F is gating strategies of CD8+ and CD4+ T cells. FIG. 9G is CD4+ T cell subclusters and cell count.

FIGS. 10A-10D show subcluster analysis of dendritic cells in mouse liver. FIG. 10A is a heatmap of top 10 cluster marker genes for DC subclusters. FIG. 10B is feature plots illustrating subcluster marker gene expression. FIG. 10C is UMAP of DC subclusters. FIG. 10D is cell count of DC subclusters.

FIGS. 11A-11D show single-cell RNAseq analysis of liver NPCs from WT and NRG4 KO mice following diet-induced NASH. FIG. 11A is UMAP representation of 16 major liver cell clusters. Single-cell RNAseq analysis of liver NPCs isolated from WT (n=4) and Nrg4 KO (n=4) mice following 7 months of NASH diet feeding. FIG. 11B is a heatmap of cluster marker gene expression. FIG. 11C is cell count for major cell types in WT and Nrg4 KO mouse livers. FIG. 11D is a heatmap of marker gene expression in macrophage subclusters. Representative markers for each subcluster are indicated on the right.

FIGS. 12A-12C show subcluster analysis of T cells in WT and NRG4 KO NASH livers. FIG. 12A is a pie chart for T cell subtypes in WT and NRG4 KO livers. FIG. 12B is a volcano plot of gene expression using averaged values of normalized expression levels for all T cells from WT and NRG4 KO livers. X-axis indicates log-transformed fold change of gene expression between NRG4 KO and WT livers. A subset of downregulated and upregulated genes are indicated on the right. FIG. 12C is virtual flow analysis of CD8+ T cells within NPCs from WT and Nrg4 KO livers by gating for Pdcd1 and Lag3 expression.

FIGS. 13A and 13B show recombinant hNRG4-Fc fusion protein inhibits oncogene-induced HCC. FIG. 13A is immunoblots of plasma Fc and hNRG4-Fc protein from mice transduced with AAV-Fc or AAV-hNRG4-Fc. FIG. 13B is graphs of metabolic parameters in male mice of 4 months of age transduced with AAV-cMYC/AAVnRAS followed by twice-weekly treatment with Fc (2.5 mg/kg, n=12) or hNRG4-Fc at 0.5 mg/kg (n=10) or 2.5 mg/kg (n=10).

FIGS. 14A and 14B show ligand and receptor network analysis. FIG. 13A is CellPhoneDB analysis of intercellular crosstalk between macrophages and CD8+ T cells. Predicted macrophage to T cell (left) and T cell to macrophage (right) ligand receptor pairs are indicated. FIG. 14B is hepatic TREM2 expression as a predictor for survival in liver cancer patients. The graph was generated based on data obtained from The Cancer Genome Atlas (TCGA).

FIG. 15 is graphs of the tumor-suppressing effects of hNRG4-Fc fusion protein in a spontaneous liver cancer model. Mice were treated with either Fc or hNRG4-Fc, as indicated, and monitored for body weight, blood glucose, maximal tumor size, and total tumor counts.

### DETAILED DESCRIPTION

Diet-induced NASH is characterized by induction of tumor-associated macrophage (TAM)-like macrophages and exhaustion of cytotoxic T cells in mouse liver. The adipocyte-derived endocrine factor Neuregulin 4 (NRG4) serves as a hormonal checkpoint that restrains this pathological reprogramming during NASH. NRG4 deficiency exacerbated the induction of tumor-prone liver immune microenvironment and NASH-related HCC, whereas transgenic NRG4 overexpression elicited protective effects in mice. Described herein is an NRG4-Fc fusion protein, and compositions thereof, that exhibit greater efficacy in receptor activation and a prolonged half-life. NRG4-Fc fusion protein potently suppressed NASH-associated liver tumorigenesis in a mouse model of NASH hepatocellular carcinoma (HCC).

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the terms “administering,” “providing,” and “introducing,” are used interchangeably herein and refer to the placement into a cell, organism, or subject by a method or route which results in at least partial localization a desired site.

As used herein, the term “chemotherapeutic” or “anti-cancer drug” includes any small molecule or other drug used in cancer treatment or prevention. Chemotherapeutics include, but are not limited to, cyclophosphamide, methotrexate, 5-fluorouracil, doxorubicin, docetaxel, daunorubicin, bleomycin, vinblastine, dacarbazine, cisplatin, paclitaxel, raloxifene hydrochloride, tamoxifen citrate, abemacicilib, afinitor (Everolimus), alpelisib, anastrozole, pamidronate, anastrozole, exemestane, capecitabine, epirubicin hydrochloride, eribulin mesylate, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, and tucatinib.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. The term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

As used herein, the terms “percent sequence identity” or “percent identity” refer to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The polypeptides may be modified by the addition of sugars, lipids or other moieties not included in the amino acid chain.

A “subject” or “patient” may be human or non-human and may include, for example, animal strains or species used as “model systems” for research purposes, such a mouse model as described herein. Likewise, subject may include either adults or juveniles (e.g., children). Moreover, subject may mean any living organism, preferably a mammal (e.g., humans and non-humans) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

A “vector” or “expression vector” is a replicon, such as plasmid, phage, virus, or cosmid, to which another DNA segment, e.g., an “insert,” may be attached or incorporated so as to bring about the replication of the attached segment in a cell.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. NRG4 POLYPEPTIDES AND Fc FUSION PROTEINS

The NRG4 polypeptide may comprise the protein encoded by the human NRG4 gene. In some embodiments, NRG4 has an amino acid sequence of SEQ ID NO: 1. NRG4 was discovered based on its sequence homology to other NRG members and is predicted to encode a precursor protein of 115 amino acids. NRG4 is highly conserved between mouse and human, with over 90% amino acid sequence identity in the EGF-like domain (approximately amino acids 1-52 of SEQ ID NO: 1). In some embodiments, the term NRG4 polypeptide refers to SEQ ID NO: 2, the protein encoded by the mouse NRG4 gene.

The NRG4 polypeptide may comprise an NRG4 variant comprising at least 70% identity to SEQ ID NO: 1. In some embodiments, the NRG4 polypeptide may comprise an NRG4 variant comprising at least 70%, at least 75%, at least 80%, at least 85%, at least 90%, at least 95%, at least 97%, at least 98%, or at least 99% identity to SEQ ID NO: 1.

The NRG4 polypeptide may comprise an NRG4 variant comprising at least 70% identity to SEQ ID NO: 2. In some embodiments, the NRG4 polypeptide may comprise an NRG4 variant comprising at least 70%, at least 75%, at least 80%, at least 85%, at least 90%, at least 95%, at least 97%, at least 98%, or at least 99% identity to SEQ ID NO: 2.
SEQ ID NO: 1—
MPTDHEEPCGPSHKSFCLNGGLCYVIPTIPSPFCRCVENYTGARCEEVFLPGSSIQTKSNL FEAFVALAVLVTLIIGAFYFLCRKGHFQRASSVQYDINLVETSSTSAHHSHEQH
   SEQ ID NO: 2—
MPTDHEQPCGPRHRSFCLNGGICYVIPTIPSPFCRCIENYTGARCEEVFLPSSSIPSESNLSA AFVVLAVLLTLTIAALCFLCRKGHLQRASSVQCEISLVETNNTRTRHSHREH

The NRG4 polypeptide may comprise a biologically active fragment of NRG4 protein or variant thereof. Biologically active fragments of the NRG4 protein include, but are not limited to, an NRG4 fragment that lacks the first 4 residues of SEQ ID NO: 1, a fragment that comprises or consists of amino acid residues 5 through 46, 5 through 55, 5 through 62, 1 through 46, 1 through 55, 1 through 52, 1 through 53, 4 through 52, 4 through 53, or 1 through 62 of SEQ ID NO: 1. A person of skill in the art can readily screen for active fragments by screening for activity in a relevant biological assay. In various embodiments, an NRG4 fragment comprises or consists of amino acid residues x to y, wherein x is 1, 2, 3, 4, 5, 6, 7, 8, 9, 10, 11, 12, 13, 14, 15, 16, 17, 18, 19, 20, 21, 22, 23, 24, or 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 and y is 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, or 115.

In some embodiments, the NRG4 polypeptide is linked to a Fc domain. Thus, the disclosure provides a fusion protein comprising a Neuregulin 4 (NRG4) polypeptide and an Fc domain. A fusion protein comprising a Neuregulin 4 (NRG4) polypeptide and an Fc domain (NRG4-Fc fusion protein) refers to a fusion protein in which a NRG4 polypeptide is linked, directly or indirectly, to an Fc domain.

The terms “Fc domain,” “Fc,” or “Fc region” are used interchangeably herein and refer to the polypeptide comprising the constant region of an antibody excluding, in some instances, the first constant region immunoglobulin domain (e.g., CH1) or a portion thereof, and in some cases, part of the hinge. Thus, an Fc domain can refer to the last two constant region immunoglobulin domains (e.g., CH2 and CH3) of IgA, IgD, and IgG, the last three constant region immunoglobulin domains of IgE and IgM, and the flexible hinge N-terminal to these domains. For IgA and IgM, Fc may include the J chain. In some embodiments, an Fc domain refers to a truncated CH1 domain, and CH2 and CH3 of an immunoglobulin. Although the boundaries of the Fc domain may vary, the human IgG heavy chain Fc domain is usually defined to include residues E216 or C226 or P230 to its carboxyl-terminus, wherein the numbering is according to the EU index as in Kabat. However, the C-terminal lysine (Lys447) of the Fc domain may or may not be present, without affecting the structure or stability of the Fc domain.

In certain embodiments, Fc domain refers to an immunoglobulin IgG heavy chain constant region comprising a hinge region (starting at Cys226), an IgG CH2 domain and CH3 domain. The term “hinge region” or “hinge sequence” as used herein refers to the amino acid sequence located between the linker and the CH2 domain. Fc region from an IgG subclass of any given animals. For example, in humans, the IgG classes including IgG1, IgG2, IgG3, and IgG4; in mouse the IgG classes including IgG1, IgG2a, IgG2b, and IgG3; and in rat the IgG classes including IgG1, IgG2a, IgG2b, IgG2c, and IgG3. It is known that certain IgG subclasses, for example, rat IgG2b and IgG2c, have higher clearance rates than, for example, IgG1. Thus, when using IgG subclasses other than IgG1 it may be advantageous to substitute one or more of the residues, particularly in the CH2 and CH3 domains, which differ from the IgG1 sequence with those of IgG1, thereby increasing the in vivo half-life of the other types of IgG. In certain particular embodiments, the Fc domain comprises the Fc domain of human IgG1, IgG2, IgG3 or IgG4. In certain other particular embodiments, the Fc domain comprises the CH2 and CH3 domain of IgG1.

In some embodiments, the Fc domain is a native sequence Fc domain. In some embodiments, amino acid modifications are made to the Fc domain, for example to alter binding to one or more receptors or to alter serum half-life, by modifying or engineering the native sequence Fc domain. The possible variants of altered Fc-fusion proteins useful with the present invention are many and range from the changing of just one or a few amino acids to the complete redesign of, for example, the constant region. Changes in the constant region will, in general, be made in order to improve, or alter (e.g., increase or decrease) characteristics, such as binding interactions with various Fc-gamma receptors and/or other immunoglobulin effector functions. In some embodiments, an Fc domain is altered to increase or decrease the extent to which the fusion protein is glycosylated. Addition or deletion of glycosylation sites to a protein may be conveniently accomplished by altering the amino acid sequence such that one or more glycosylation sites is created or removed. In certain embodiments, one or more residues of the Fc domain are substituted with cysteine residues, to position reactive thiol groups at accessible sites of the Fc domain, for use in conjugating the Fc domain to other moieties.

The NRG4 polypeptide and the Fc domain may be linked in any orientation. In some embodiments, the N-terminus of the NRG4 polypeptide is linked to the C-terminus of the single subunit of the Fc domain. In some embodiments, the C-terminus of the NRG4 polypeptide is linked to the N-terminus of the single subunit of the Fc domain. In some embodiments, the N-terminus of the NRG4 polypeptide is linked to the N-terminus of the single subunit of the Fc domain. In some embodiments, the C-terminus of the NRG4 polypeptide is linked to the C-terminus of the single subunit of the Fc domain.

In some embodiments, the NRG4-Fc fusion protein comprises a linker between the NRG4 polypeptide and the Fc domain. The linker may have any of a variety of amino acid sequences and be a variety of lengths (e.g., 4-100 amino acids). The linker can be produced by using synthetic, linker-encoding oligonucleotides to couple the portions of the fusion protein or can be encoded by a nucleic acid sequence encoding the fusion protein. In some embodiments, the linker polypeptide is considered a flexible linker, facilitating some degree of orientation freedom for NRG4 polypeptide and the Fc domain. A variety of different linkers are considered suitable for use, including but not limited to, glycine-serine polymers, glycine-alanine polymers, and alanine-serine polymers.

Any of the NRG4 polypeptides or the Fc domains described herein may comprise one or more (e.g., 1, 2, 3, 4, 5, 6, 7, 8, 9, 10, 15, 20, 25, 50, 100, etc.) amino acid substitutions. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free-OH can be maintained, and glutamine for asparagine such that a free-NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In some embodiments, the NRG4 polypeptide comprises one or more lipid moieties. Lipids which can be covalently attached to proteins include, for example, fatty acids, isoprenoids, sterols, and phospholipids. The lipid moieties may be directly or indirectly attached at any part of the NRG4 polypeptide. The attachment of the lipid moieties may improve the half-life of the NRG4 polypeptide or NRG4-Fc fusion protein, for example in plasma, and thus increase its overall efficacy as compared to a NRG4 polypeptide or NRG4-Fc fusion protein lacking the lipid moiety.

### a. Nucleic Acids

Also provided herein are nucleic acids encoding the NRG4 polypeptide or NRG4-Fc fusion protein. In some embodiments, the NRG4 polypeptide or NRG4-Fc fusion protein may be encoded by a nucleic acid (e.g., a vector) configured to be introduced into the cell. In some embodiments, the nucleic acid is DNA. In some embodiments, the nucleic acid is RNA.

Nucleic acids encoding the NRG4 polypeptide or NRG4-Fc fusion protein described herein can comprise any of a number of promoters, including, but not limited to, constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, mycoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible expression can be accomplished by placing the nucleic acid encoding such a molecule under the control of an inducible promoter/regulatory sequence. Promoters that are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence that is capable of driving expression of the desired protein operably linked thereto.

The present disclosure also provides for vectors containing the nucleic acids and cells containing the nucleic acids or vectors, thereof. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

The vectors of the present disclosure may direct the expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene for selection of stable or transient transfectants in host cells; transcription termination and RNA processing signals; 5′- and 3′-untranslated regions; internal ribosome binding sites (IRESes), versatile multiple cloning sites; and reporter gene for assessing expression of the chimeric receptor. Suitable vectors and methods for producing vectors containing transgenes are well known and available in the art. Selectable markers include chloramphenicol resistance, tetracycline resistance, spectinomycin resistance, neomycin, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into a cell, the vectors may be maintained as an autonomously replicating sequence or extrachromosomal element or may be integrated into host DNA.

Conventional viral and non-viral based gene transfer methods can be used to introduce the nucleic acids into cells, tissues, or a subject. Such methods can be used to administer the nucleic acids to cells in culture, or in a host organism. Non-viral vector delivery systems include DNA plasmids, cosmids, RNA (e.g., a transcript of a vector described herein), a nucleic acid, and a nucleic acid complexed with a delivery vehicle.

Viral vector delivery systems include DNA and RNA viruses, which have either episomal or integrated genomes after delivery to the cell. A variety of viral constructs may be used to deliver the present nucleic acids to the cells, tissues and/or a subject. Viral vectors include, for example, retroviral, lentiviral, adenoviral, adeno-associated, baculoviral, and herpes simplex viral vectors. Nonlimiting examples of such recombinant viruses include recombinant adeno-associated virus (AAV), recombinant adenoviruses, recombinant lentiviruses, recombinant retroviruses, recombinant herpes simplex viruses, recombinant baculoviruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1): 33-40; and Walther W. and Stein U., 2000 Drugs, 60(2): 249-71, incorporated herein by reference.

Vectors according to the present disclosure can be transformed, transfected, or otherwise introduced into a wide variety of cells. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Methods of delivering vectors to cells may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110 (6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell.

Additionally, delivery vehicles such as nanoparticle-and lipid-based delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1; 459(1-2): 70-83), incorporated herein by reference.

### b. Compositions

Further disclosed herein are compositions comprising a NRG4 polypeptide or NRG4-Fc fusion protein or nucleic acids encoding thereof as described above. The compositions may further comprise excipients or pharmaceutically acceptable carriers. The choice of excipients or pharmaceutically acceptable carriers will depend on factors including, but not limited to, the particular mode of administration, the effect of the excipient on solubility and stability, and the nature of the dosage form.

Excipients and carriers may include any and all solvents, dispersion media, antibacterial and antifungal agents, isotonic and absorption delaying agents. Some examples of materials which can serve as excipients and/or carriers are sugars including, but not limited to, lactose, glucose and sucrose; starches including, but not limited to, com starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, preservatives, and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995).

The compositions may be formulated for any appropriate manner of administration, and thus administered, including for example, oral, nasal, intravenous, intravaginal, epicutaneous, sublingual, intracranial, intradermal, intraperitoneal, subcutaneous, intramuscular administration, or via inhalation. The compositions may be suitable for implantation, intramuscularly or subcutaneously, as depot injectors or as implants.

Techniques and formulations may generally be found in “Remington's Pharmaceutical Sciences,” (Meade Publishing Co., Easton, Pa.). Therapeutic or pharmaceutical compositions must typically be sterile and stable under the conditions of manufacture and storage. The route or administration and the form of the composition usually dictates the type of carrier to be used.

The compositions may also comprise buffers (e.g., neutral buffered saline or phosphate buffered saline), carbohydrates (e.g., glucose, mannose, sucrose or dextrans), mannitol, antioxidants, bacteriostats, chelating agents such as EDTA or glutathione, solutes that render the formulation isotonic, hypotonic, or weakly hypertonic with the blood of a recipient, suspending agents, thickening agents and/or preservatives, commonly found in proteinaceous compositions.

The disclosed fusion proteins or nucleic acids may be in entrapped in microcapsules, in colloidal drug delivery systems (for example, liposome, albumin microspheres, microemulsions, nano-particles and nanocapsules), in macroemulsions, or in sustained-release preparation. The disclosed fusion proteins or nucleic acids may be in a liposome and combined with amphipathic agents such as lipids which exist in aggregated form as micelles, insoluble monolayers, liquid crystals, or lamellar layers which in aqueous solution. Suitable lipids for liposomal formulations include, without limitation, monoglycerides, diglycerides, sulfatides, lysolecithin, phospholipids, saponin, and bile acids. Preparation of such liposomal formulations is within the level of skill in the art.

In some embodiments, the compositions comprise one or more additional therapeutic agents. The additional one or more therapeutic agents may comprise one or more chemotherapeutic agents. In some embodiments, the one or more additional therapeutic agents comprise a checkpoint inhibitor, a receptor tyrosine kinase inhibitor, or a combination thereof.

Immune checkpoint molecules are negative regulators of immune responses which prevent the immune system from attacking cells indiscriminately. Known immune checkpoint molecules include, for example, CTLA-4, PD-1, PD-L1 (programmed Cell death-ligand 1), PD-L2 (programmed Cell death-ligand 2), LAG-3 (lymphocyte activation gene 3), TIM3 (T-Cell immunoglobulin and mucin-3), BTLA (B and T-lymphocyte attenuators), B7H3, B7H4, CD160, CD39, CD73, A2aR (adenosine A2a receptor), KIR (killer inhibitory receptor), VISTA (V-domain Ig-containing inhibitor of T-Cell activation), IDO1 (indoleamine 2, 3-dioxygenase), arginase I, TIGIT (T-Cell immunoglobulin and ITIM domain 2015), and CD115. The immune checkpoint inhibitors useful in the methods disclosed herein are substances that inhibit the function of immune checkpoint molecules. For example, CTLA4 checkpoint inhibitors include, without limitation, monoclonal antibodies such as ipilimumab and tremelimumab and PD-1/PD-L1 checkpoint inhibitors include, without limitation, monoclonal antibodies against PD-1 such as nivolumab, pembrolizumab, atezolizumab, and pidilizumab, anti-PD-1 fusion proteins such as AMP-224 (composed of the extracellular domain of PD-L2 and the Fc region of human IgG1), and monoclonal antibodies against PD-L1 such as BMS-936559 (MDX-1105), atezolizumab, durvalumab, and avelumab.

Tyrosine kinase inhibitors are compounds that inhibit or block the activity of tyrosine kinase enzymes. These enzymes can phosphorylate many regulatory proteins in the cell and can activate signal transduction cascades, triggering many cellular functions involving cell growth and proliferation. There are two types of tyrosine kinases: cell surface receptor protein kinases (RTKs) and non-receptor protein kinases (NRTKs). Receptor tyrosine kinases belong to the family of cell surface receptors that transduce a response upon binding to a ligand. They are transmembrane proteins that pass through the biological membrane and have an extracellular domain (ectodomains) where ligands can bind. Examples of RTKs include, but are not limited to, Vascular Endothelial Growth Factor Receptor (VEGFR), Epidermal Growth Factor Receptor (EGFR), Platelet-Derived Growth Factor Receptor (PDGFR), and Fibroblast Growth Receptor (FGR). Non-receptor tyrosine kinases are located within the cytosol, they are activated upon binding to an already activated receptor tyrosine kinase receptor and are accountable for the activation of receptor by phosphorylation without the presence of a ligand. Examples of NRTKs include, but are not limited to, v-SRC (Rous sarcoma virus), Bcr-Abl (Abelson protooncogene-breakpoint cluster region) fusion.

Receptor tyrosine kinase inhibitors can either be monoclonal antibodies that compete for the receptor's extracellular domain or small molecules that inhibit the tyrosine kinase domain and prevent conformational changes that activate RTKs. In some embodiments, the receptor tyrosine kinase inhibitor may be an antibody including, for example, a monoclonal antibody. Monoclonal antibodies may include, but are not limited to cetuximab, panitumumab, zalutumumab, nimotuzumab, bevacizumab, or matuzumab. In other embodiments, the receptor tyrosine kinase inhibitor is a small molecule inhibitor. Small molecule inhibitors may include, but are not limited to, sorafenib, lenvatinib, regorafenib, sunitinib, apatinib, donfenib, anlotinib, and cabozantinib.

### 3. METHODS

The present disclosure provides methods for treating, reducing, or preventing cancer, e.g., treating a subject or in vitro treatment of cancer cells isolated from a subject or from a cancer cell line.

In some embodiments, the methods comprise introducing into the cell or administering to a subject an effective amount of a NRG4 polypeptide or a fusion protein comprising a Neuregulin 4 (NRG4) polypeptide and an Fc domain, a nucleic acid encoding thereof, or a composition thereof, as described above. An “effective amount” is an amount that is delivered, either in a single dose or as part of a series, which is effective for inducing a response.

When utilized as a method of treatment in a subject, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. It is expected that the amount will fall in a relatively broad range that can be determined by one of skill in the art through routine trials.

In some embodiments, the methods are for treating, reducing, or preventing cancer in a subject in need thereof. In some embodiments, the subject is a human. In some embodiments, the subject has cancer, has had cancer, is predisposed to cancer, is suspected of having cancer, or has a family history of cancer.

In some embodiments, the cancer is invasive and/or metastatic cancer (e.g., stage II cancer, stage III cancer or stage IV cancer). In some embodiments, the cancer is an early stage cancer (e.g., stage 0 cancer, stage I cancer), and/or is not invasive and/or metastatic cancer.

In some embodiments, the methods result in decreased tumor growth. In some embodiments, the methods result in tumor regression. In some embodiments, the methods result in decreased numbers of tumor. In some embodiments, the methods result in decreased tumor growth. In some embodiments, the methods prevent tumor recurrence. In some embodiments, the methods result in increases in overall subject survival.

The methods herein may be useful to treat a wide variety of cancers including carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, lymph nodes, muscle tissue, ovary, pancreas, prostate, skin, spleen, stomach, testicle, thyroid, or uterus. In some embodiments, the cancer comprises a solid tumor.

In some embodiments, the cancer is liver cancer. In some embodiments, the liver cancer is intermediate, advanced, or terminal stage. The liver cancer can be metastatic or non-metastatic. The liver cancer may be resectable or unresectable. The liver cancer may include a single tumor, multiple tumors, or a poorly defined tumor with an infiltrative growth pattern (into portal veins or hepatic veins). The liver cancer may include a fibrolamellar, pseudoglandular (adenoid), pleomorphic (giant cell), or clear cell pattern. The liver cancer may include a well differentiated form, and tumor cells resemble hepatocytes, form trabeculae, cords, and nests, and/or contain bile pigment in cytoplasm. The liver cancer may include a poorly differentiated form, and malignant epithelial cells are discohesive, pleomorphic, anaplastic, and/or giant.

In some embodiments, the liver cancer may be associated with hepatic steatosis, particularly nonalcoholic steatohepatitis (NASH). In certain embodiments, the liver cancer is NASH-associated liver cancer. Thus, in some embodiments, the subject may be suffering from hepatic steatosis or NASH.

The liver cancer may be a hepatocellular carcinoma (HCC), a hepatoblastoma, a cholangiocarcinoma, a cholangiocellular cystadenocarcinoma, an angiosarcoma, a hemangioendothelioma, or a combination thereof.

The most frequent liver cancer, accounting for approximately 75% of all primary liver cancers, is hepatocellular carcinoma (HCC). HCC is a cancer formed by liver cells, known as hepatocytes, that become malignant. Another type of cancer formed by liver cells is hepatoblastoma, which is specifically formed by immature liver cells. It is a rare malignant tumor that primarily develops in children, and accounts for approximately 1% of all cancers in children and 79% of all primary liver cancers under the age of 15.

Liver cancer can also form from other structures within the liver such as the bile duct, blood vessels and immune cells. Cancer of the bile duct (cholangiocarcinoma and cholangiocellular cystadenocarcinoma) account for approximately 6% of primary liver cancers. There is also a variant type of HCC that consists of both HCC and cholangiocarcinoma. Tumors of the liver blood vessels include angiosarcoma and hemangioendothelioma. Embryonal sarcoma and fibrosarcoma are produced from a type of connective tissue known as mesenchyme. Cancers produced from muscle in the liver are leiomyosarcoma and rhabdomyosarcoma. Other less common liver cancers include carcinosarcomas, teratomas, yolk sac tumors, carcinoid tumors and lymphomas. Lymphomas usually have diffuse infiltration to liver, but it may also form a liver mass in rare occasions.

In some embodiments, the liver cancer is primary liver cancer, which is a disease in which cancer (malignant) cells start to grow in the tissues of the liver. The liver is a popular target for metastasis since it receives blood from the abdominal organs via the portal vein. In some embodiments, the liver cancer is metastatic liver cancer in which malignant cells originated in another organ (e.g., lung, breast, colon, rectum). The liver cancer can be at any stage of progression.

Diagnosis of liver cancer is based on a combination of ultrasonography, fine-needle biopsy, and detection of circulating levels of certain marker proteins, including, for example, alpha-fetoprotein. Liver cancer, e.g., HCC, can be classified into early, intermediate, advanced, and end-stage cancer based on tumor size, number, and morphology (e.g., encapsulated or invasive), and liver function. In some embodiments, the subject has been diagnosed with liver cancer. The liver cancer can be a first occurrence or a recurrence. Thus, suitable subjects also include those previously treated for liver cancer, who, after a period of remission, have recurring liver cancer.

The NRG4 polypeptide, NRG4-Fc fusion protein, a nucleic acid encoding thereof, or a composition thereof, may be administered to a subject by a variety of methods. In any of the uses or methods described herein, administration may be by various routes known to those skilled in the art, including without limitation oral, inhalation, intravenous, intramuscular, topical, subcutaneous, systemic, and/or intraperitoneal administration to a subject in need thereof.

The amount of the NRG4 polypeptide, NRG4-Fc fusion protein, or a composition thereof, of the present disclosure required for use in treatment or prevention will vary not only with the particular compound selected but also with the route of administration, the nature and/or symptoms of the cancer and the age and condition of the patient and will be ultimately at the discretion of the attendant physician or clinician. The determination of effective dosage levels, that is the dosage levels necessary to achieve the desired result, can be accomplished by one skilled in the art using routine methods, for example, human clinical trials, in vivo studies, and in vitro studies. For example, useful dosages of a NRG4 polypeptide, NRG4-Fc fusion protein, or a composition thereof, can be determined by comparing their in vitro activity, and in vivo activity in animal models.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. Bioassays can be used to determine plasma concentrations. Dosage intervals can also be determined using MEC value. Compositions should be administered using a regimen, which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

A NRG4 polypeptide, NRG4-Fc fusion protein, a nucleic acid encoding thereof, or a composition thereof, as disclosed herein can be evaluated for efficacy and toxicity using known methods. For example, the toxicology NRG4-Fc fusion protein, a nucleic acid encoding thereof, or a composition thereof, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs, or monkeys, may be determined using known methods. Efficacy may be established using several recognized methods, such as in vitro methods, animal models, or human clinical trials. When selecting a model to determine efficacy, the skilled artisan can be guided by the state of the art to choose an appropriate model, dose, route of administration and/or regime.

A wide range of second therapies may be used in conjunction with the compounds of the present disclosure. The second therapy may be administration of an additional therapeutic agent or may be a second therapy not connected to administration of another agent. Such second therapies include, but are not limited to, surgery, immunotherapy, radiotherapy, or a chemotherapeutic or anti-cancer agent.

The second therapy may be administered at the same time as the initial therapy, either in the same composition or in a separate composition administered at substantially the same time as the initial composition. In some embodiments, the second therapy may precede or follow the treatment of the first therapy by time intervals ranging from hours to months.

In some embodiments, a therapeutically effective amount of a NRG4 polypeptide, a fusion protein comprising a Neuregulin 4 (NRG4) polypeptide and an Fc domain or a nucleic acid encoding thereof, or compositions thereof, is administered alone or in combination with a therapeutically effective amount of at least one additional therapeutic agent. In some embodiments, effective combination therapy is achieved with a single composition or pharmacological formulation that includes the additional therapeutic agent(s), or with two distinct compositions or formulations, administered at the same time or separated by a time interval, wherein one composition includes a NRG4 polypeptide or a NRG4-Fc fusion protein, or a nucleic acid encoding thereof, and the other includes the at least one additional therapeutic agent.

In some embodiments, the second therapy includes immunotherapy. Immunotherapies include chimeric antigen receptor (CAR) T-cell or T-cell transfer therapies, cytokine therapy, immunomodulators, cancer vaccines, or administration of antibodies (e.g., monoclonal antibodies).

In some embodiments, the immunotherapy comprises administration of antibodies. The antibodies may target antigens either specifically expressed by tumor cells or antigens shared with normal cells. In some embodiments, the immunotherapy may comprise an antibody targeting, for example, CD20, CD33, CD52, CD30, HER (also referred to as erbB or EGFR), VEGF, CTLA-4 (also referred to as CD152), epithelial cell adhesion molecule (EpCAM, also referred to as CD326), and PD-1/PD-L1. Suitable antibodies include, but are not limited to, rituximab, blinatumomab, trastuzumab, gemtuzumab, alemtuzumab, ibritumomab, tositumomab, bevacizumab, cetuximab, panitumumab, ofatumumab, ipilimumab, brentuximab, pertuzumab and the like). In some embodiments, the additional therapeutic agent may comprise anti-PD-1/PD-L1 antibodies, including, but not limited to, pembrolizumab, nivolumab, cemiplimab, atezolizumab, avelumab, durvalumab, and ipilimumab. The antibodies may also be linked to a chemotherapeutic agent. Thus, in some embodiments, the antibody is an antibody-drug conjugate.

The immunotherapy (e.g., administration of antibodies) may be administered to a subject by a variety of methods. In any of the uses or methods described herein, administration may be by various routes known to those skilled in the art, including without limitation oral, inhalation, intravenous, intramuscular, topical, subcutaneous, systemic, and/or intraperitoneal administration to a subject in need thereof. In some embodiments, the immunotherapy may be administered in the same or different manner than the JAK inhibitor analog, or composition thereof. The immunotherapy may be administered by parenteral administration (including, but not limited to, subcutaneous, intramuscular, intravenous, intraperitoneal, intracardiac and intraarticular injections).

In some embodiments, the at least one additional therapeutic agent comprises a checkpoint inhibitor, a receptor tyrosine kinase inhibitor, or a combination thereof.

The immune checkpoint inhibitors suitable for use in the methods disclosed herein are substances that inhibit the function of immune checkpoint molecules, as described elsewhere herein. For example, CTLA4 checkpoint inhibitors include, without limitation, monoclonal antibodies such as ipilimumab and tremelimumab, and PD-1/PD-L1 checkpoint inhibitors include, without limitation, monoclonal antibodies against PD-1 such as nivolumab, pembrolizumab, atezolizumab, and pidilizumab, anti-PD-1 fusion proteins such as AMP-224 (composed of the extracellular domain of PD-L2 and the Fc region of human IgG1), and monoclonal antibodies against PD-L1 such as BMS-936559 (MDX-1105), atezolizumab, durvalumab, and avelumab.

Tyrosine kinase inhibitors are compounds that inhibit or block the activity of tyrosine kinases: cell surface receptor protein kinases (RTKs) and non-receptor protein kinases (NRTKs), as described elsewhere herein. Receptor tyrosine kinase inhibitors can either be monoclonal antibodies that compete for the receptor's extracellular domain or small molecules that inhibit the tyrosine kinase domain and prevent conformational changes that activate RTKs. In some embodiments, the receptor tyrosine kinase inhibitor may be an antibody including, for example, a monoclonal antibody. Monoclonal antibodies may include, but are not limited to cetuximab, panitumumab, zalutumumab, nimotuzumab, bevacizumab, or matuzumab. In other embodiments, the receptor tyrosine kinase inhibitor is a small molecule inhibitor. Small molecule inhibitors may include, but are not limited to, sorafenib, lenvatinib, regorafenib, sunitinib, apatinib, donfenib, anlotinib, and cabozantinib.

### 4. KITS

In another aspect, the disclosure provides kits comprising a NRG4 polypeptide, NRG4-Fc fusion protein, a nucleic acid encoding thereof, or a composition thereof, and instructions for using the compound or composition.

The kits can also comprise other agents and/or products co-packaged, co-formulated, and/or co-delivered with other components. For example, a drug manufacturer, a drug reseller, a physician, a compounding shop, or a pharmacist can provide a kit comprising a disclosed compound and/or product and another agent (e.g., a chemotherapeutic, an immunotherapy) for delivery to a patient. In some embodiments, the kits further comprise a checkpoint inhibitor, a receptor tyrosine kinase inhibitor, or a combination thereof.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

The kits provided herein are in suitable packaging. Suitable packaging includes, but is not limited to, vials, bottles, jars, flexible packaging, and the like. Individual member components of the kits may be physically packaged together or separately.

It is understood that the disclosed kits can be employed in connection with the disclosed methods. The kit may further contain containers or devices for use with the methods or compositions disclosed herein, for example delivery devices (e.g., syringes and the like).

### 5. EXAMPLES

The following are examples of the present invention and are not to be construed as limiting.

### Materials and Methods

Human study Normal or NASH human liver tissues were obtained from the Liver Tissue Cell Distribution System at the University of Minnesota (Minneapolis, Minnesota, USA). Both male and female individuals were included. The average ages for normal individuals and NASH patients were 55.8 and 52.5 years old, respectively. Individuals with an alcohol-drinking history (2 to 3 drinks/day) and liver cancer were excluded from the study.

Animal studies Mice were housed in pathogen-free facilities under 12-h light-dark cycles with free access to food (Teklad 5001 Laboratory Diet) and water. Nrg4 KO mice were generated at the University of Michigan Transgenic Animal Model Core using Nrg4 tm1a(EUCOMM)Hmgu ES cells purchased from the International Mouse Phenotyping Consortium. Whole body Nrg4 KO mice were generated by crossing Nrg4 flox mice with Ella-Cre mice (a gift from Dr. David Ginsburg, University of Michigan). Trem2 KO mice were purchased from The Jackson Laboratory (Strain #: 027197). OT-I transgenic mice and Rosa26-tdTomato reporter mice were kindly provided by Dr. Weiping Zou and Dr. Jun Wu (University of Michigan), respectively. Trem2-Cre knockin mouse strain that contains Cre recombinase fused to endogenous Trem2 via the self-cleavage P2A peptide was generated. Trem2-Cre mice were crossed with the Rosa26-tdTomato reporter strain to label the Trem2-expressing macrophage lineage. Adipose-specific Nrg4 transgenic mice were generated and described previously (Wang et al., 2014).

Sample size was not predetermined, group sizes typical for this type of work in the literature were used. Mice of the same genotype were randomly assigned to different treatments to minimize any potential bias. The investigators were not blinded to allocation during experiments and outcome assessment. Mice that exhibited skin lesions due to fighting and growth retardation as a result of malocclusion were excluded.

NPC isolation, BMDM culture and cell lines For hepatic macrophages, liver samples were filtered through 100 μm strainers in 1% FBS, 1 mM EDTA in PBS and centrifuged at 50 g for 3 minutes to remove hepatocytes. For hepatic T cells, liver tissues were minced briefly and immersed in 3 mL of RPMI 1640 plus 2 mg/mL collagenase IV and 0.1 mg/mL DNase I at 37° C. for 20 minutes with periodic agitation. Liver tissue was then filtered through a 100 μm strainer and spun at 50 g for 3 minutes to remove hepatocytes. NPCs were harvested as intermediate fraction following gradient centrifugation in 25% optiprep at 1,500 g for 20 minutes. Cells were then treated with 0.8% NH4Cl for 5 minutes to lyse red blood cells. CD8+ T cells were enriched with negative selection kit (Miltenyi, 130-096-495) following manufacturer's instruction.

BMDM were differentiated from bone marrow cells harvested from the epiphyses of tibia and femur bones from 6- to 8-week-old mice. Bone marrow cell suspension was filtered through a 70 μm cell strainer, centrifuged at 250 g for 5 minutes. Cells pellets were resuspended in DMEM and treated with 0.8% NH₄Cl for 5 minutes to lyse red blood cells. BMDM was cultured in DMEM supplemented with 10% bovine growth serum, 100 μg/mL penicillin, 100 ug/mL streptomycin, and 25 ng/ml M-CSF (BioLegend). Following seven days of culture, BMDMs were treated with 2.5 ng/mL TGFβ for 24 hours.

Suspension cell line, Expi293F™, was purchased from Thermo Fisher Scientific and cultured with Expi293™ Expression Medium in Corning spinner flask at 37° C. incubator with humidified atmosphere of 8% CO₂. Min6 cells stably expressing ErbB4 were a gift from Dr. Peter Dempsey (University of Michigan), and were cultured in DMEM supplemented with 15% FBS, 1.7 g/500 mL sodium bicarbonate, 2.5 uL/500 mL β-mercaptoethanol and 1% Pen/Strep. Before conditioned media treatment (for 15 min), the cells were starved in serum-free DMEM for 4 hrs.

NASH models For diet-induced NASH, mice were fed a diet containing 40 kcal % fat, 20 kcal % fructose, and 2% cholesterol (Amylin diet, D09100310, Research Diets Inc.). In a separate diet-induced NASH model, C57BL/6 mice were maintained on Choline-Deficient, Amino acid-defined HFD (45 kcal % fat) containing 0.1% methionine (CDA-HFD diet, A06071309, Research Diets Inc.) for 6 weeks.

HCC models, treatment, and analysis For DEN/NASH HCC model, male pups were injected with a single i.p. dose of DEN (25 mg/kg body weight, Millipore-Sigma) on postpartum day 15. Mice were switched to NASH diet at two months of age and analyzed 5-6 months later. For quantification, HCC tumor numbers and the largest tumor sizes were determined by counting the number of visible tumors and measuring the size of the largest tumor with a caliper, respectively. Plasma concentrations of ALT, AST and cholesterol were measured using commercial assay kits (Stanbio Laboratory). Plasma concentrations of TAG was measured using Sigma kits. For oncogene NASH HCC model, male mice were put on NASH diet for two weeks before a single tail vein injection of the AAV-oncogene cocktail (AAV8-cMyc 6×10⁹ genome copies/mouse+AAV8-nRAS-V12 6×10⁹ genome copies/mouse). Two weeks after tumor induction, mice received weekly treatments of Fc or hNRG4-Fc at a dose of 0.5 or 2.5 mg/kg for four weeks before tumor analysis. For survival experiment, male mice were injected 9×10⁹ genome copies/mouse AAV8-cMyc and 9×10⁹ genome copies/mouse AAV8-nRAS-V12 via tail vein. Two weeks following transduction, mice were treated with Fc or hNRG4-Fc (1.5 mg/kg) weekly through intraperitoneal injection for a total of four weeks and monitored until termination. For hNRG4-Fc and anti-PDL1 co-treatment, C57BL/6 mice were fed NASH diet for two weeks and then injected the AAV-oncogene cocktail. At week 4, 100 μg isotype control (IgG2b) or anti-PDL1 was administered intraperitoneally to each mouse twice per week; 1.5 mg/kg Fc or hNRG4-Fc was intraperitoneally injected to mice weekly. This treatment schedule lasted four weeks and tumor burden was analyzed two weeks after the treatments. For Trem2 KO mice treatment, after two weeks of NASH diet feeding, WT and Trem2 KO mice were injected the AAV-oncogene cocktail. Two weeks later, 1.5 mg/kg Fc or hNRG4-Fc was intraperitoneally injected to mice weekly for four weeks. Tumor burden was analyzed two weeks after the treatments.

Bulk RNA sequencing and gene expression analysis Total RNA was extracted from frozen livers or harvested cells using Trizol (Alkali Scientific, TRZ-100). Bulk liver RNA sequencing was performed by BGI Global Genomic Services. The RNA-seq reads alignment was performed using the STAR aligner (version 2.7.4a) against the mouse genome assembly release mouse_GRCm38.p6 from NCBI and gene annotation release M25 from GENCODE. The mapped reads for each gene were quantified using the featureCounts function from the Rsubread package (version 2.0.1) in the R environment (version 4.0.0). Differences in transcript abundance between different genotypes were calculated for each gene using the R package DESeq2 (version 1.30.0). Genes with adjusted P-value<0.05 were considered differentially expressed. Quantitative RT-PCR gene expression analysis was performed as previously described (Li et al., 2008 Cell Metab 8, 105-117).

Isolation and scRNA-seq analysis of liver NPCs Liver NPCs were isolated following a two-step protocol of pronase/collagenase digestion. Briefly, the liver was perfused in situ with calcium-free Hank's Balanced Salt Solution (HBSS) containing 0.2 mg/mL EDTA, followed by sequential perfusion with 0.4 mg/mL pronase (Sigma, P5147) and 0.2% collagenase type II (Worthington, LS004196). The liver was minced and further digested with HBSS containing 0.2% collagenase type II, 0.4 mg/mL pronase and 0.1 mg/mL DNase I (Roche, R104159001) in 37° C. water bath with shaking for 20 min. Digestion was terminated with DMEM containing 10% serum. The resulting liver cell suspension was centrifuged at 50 g for 3 min to remove hepatocytes and passed through a 30 μm nylon cell strainer followed by treatment with 0.8% NH4Cl to lyse red blood cells. This resulting cell suspension was centrifuged, dissociated in HBSS, and subjected to density gradient centrifugation using 20% Optiprep (Axis Shield, 1114542) to remove dead cells. Cell viability was confirmed by trypan blue exclusion. The resulting NPC were subjected to scRNA-seq analysis using 10X Genomics Chromium Single-Cell 3′ according to the manufacturer's instructions at the University of Michigan Advanced Genomics Core.

Bone marrow transplantation Bone marrow cells were acquired from the femurs of donor (45.1) with Hank's buffered salt solution without calcium or magnesium, supplemented with 2% heat-inactivated calf serum (HBSS; Invitrogen). Cells were triturated and filtered through nylon screen (70 μm; Sefar America) to obtain a single-cell suspension. Recipient B6 mice (CD45.2) were irradiated in an Orthovoltage X-ray source delivering 300 rad min-1 in two equal doses of 540 rad, delivered 2 h apart. Cells were injected into intravenously through the tail. 6 weeks post-transplant blood was obtained from the tail veins of recipient mice, subject to ammonium-chloride potassium red cell lysis, and stained to monitor engraftment with CD45.2 (104), CD45.1 (A20), Cd11b (M1/80), Gr-1 (8C5).

Flow cytometry 1×10⁶ liver NPC cells were incubated with 100 μl of various antibodies diluted at optimal concentrations for 40 min at 4° C. The following fluorochrome-conjugated antibodies were used: CD45.2 (104; Biolegend, 109831), CD45.1 (A20; Biolegend, 110741), CD45 (30-F11; Biolegend, 103126) F4/80 (BM8; Biolegend, 123114), CD11b (M1/70; Biolegend, 101227), CD9 (MZ3; Biolegend, 124805), GPNMB (CSTREVL; Thermo Fisher, 50-5708-82), CD90.2 (30-H12; Biolegend, 105335), CD4 (GK1.5; Biolegend, 100407), CD8 (53-6.7; Biolegend, 100734), PD-1 (29F.1A12; Biolegend, 135218), IL-2 (JES6-5H4; Biolegend, 503807). Liver macrophages were gated as CD45+F4/80^{hi}CD11b^{int} and CD45+F4/80^{int}CD11b^{hi} for KC or MDM, respectively. For intracellular staining, 1×10⁶ NPCs were cultured in complete RMPI 1640 medium with Brefeldin A and PMA/ionomycin for 6 hours before harvested for flow staining. After surface staining, fixed cells were permeabilized using Invitrogen transcription factor staining buffer set according to manufacturer's protocol. Samples were analyzed using BD LSR cell analyzer at the Vision Research Core Facility at the University of Michigan Medical School or Attune NXT4 Flow Cytometer at MCDB research core facility at the University of Michigan. Data were analyzed using the CellQuest software (BD Biosciences) or Attene NXT software and Flowjo (Flowjo.com).

T cell proliferation assay Spleen from OT-I mice were crush through 70 um cell strainers with complete RPMI 1640. Splenocytes were pelleted and then treated with 0.8% NH4Cl for 5 minutes to lyse red blood cells. CD8+ T cells were enriched with negative selection kit (Miltenyi, 130-096-495) per manufacture's instruction. CD8+ T cells were counted and labeled with CFSE Cell Division Tracker Kit (Biolegend, 423801). Fully differentiated BMDM cells were scraped off from culture dish, and reseeded into 96 well plate at the density of 1×10⁵ cell per well. Two hours after seeding, BMDM were pulsed with OVA257-264 peptide at indicated concentration for another 2 hours. Cells were washed three times with RPMI 1640 and then 1×10⁵ labeled OT-I CD8+T cells were added and cultured for 5 days. Cell proliferation rate was determined by flow cytometry.

For intrahepatic T cell proliferation, NPC cells were harvested from liver tissue and labeled with CFSE Cell Division Tracker Kit (Biolegend, 423801). Labeled cells were transferred to 96 well plates and stimulated with CD3/CD28 Dynabeads for up to 5 days. Cell proliferation rate was determined by flow cytometry.

Immunofluorescence staining and tissue histology Liver tissues were fixed in situ with 4% paraformaldehyde, incubated with 30% sucrose in PBS overnight, and embedded in OCT. Frozen sections were permeabilized with 0.3% Triton X-100 in PBS and then blocked in 5% BSA, followed by incubation in primary antibody solution overnight at 4° C., and subsequently in secondary antibody solution at room temperature for one hour. Sections were mounted in VECTASHIELD Antifade Mounting Medium (Vector Laboratories, H-1000). Images were taken with Leica SP5 Confocal Imaging System and SP8 Lightning Confocal Microscope System. For histology, formalin-fixed, paraffin-embedded mouse liver sections were stained with H&E to evaluate steatosis and inflammatory cell infiltration. Liver fibrosis was assessed by Picrosirius (sirius) red (Polysciences, catalog 24901) staining of the formalin-fixed, paraffin-embedded mouse liver sections.

Hydroxyproline assay Collagen content in the livers was evaluated by measuring the hydroxyproline level in the livers using the Hydroxyproline Colorimetric Assay Kit (K555-100) from BioVision. Briefly, liver tissue was homogenized in water and samples were hydrolyzed by incubation with 6N hydrochloric acid at 120° C. for 3 hours. Liver hydrolysates were oxidized using chloramine-T, followed by incubation with Ehrlich's perchloric acid reagent for color development. Absorbance was measured at 560 nm, and hydroxyproline quantities were calculated by reference to standards processed in parallel.

Construction, generation, and purification of hNRG4-Fc fusion protein The NRG4-Fc fusion construct contains an N-terminal signal peptide from azurocidin 1 followed by the EGF-like domain of human NRG4 (amino acids 1-55), a glycine-serine linker and human IgG1 Fc fragment. The construct was synthesized by GeneArt (Thermo Fisher Scientific) and subcloned into pcDNA3 expression vector. For fusion protein production, the Fc vector and NRG4-Fc constructs were transiently transfected into suspension Expi293F™ cells using the Expi293 Expression System (Thermo Fisher Scientific). Media was collected 7 days after transfection, adjusted to the composition of binding buffer (0.2 M sodium phosphate, pH 7.0), filtered through a 0.45-μm filter (Millipore), and processed for affinity purification using a Hitrap rProtein A FF 5 mL column on the ÄKTA Pure FPLC chromatography system (GE Healthcare). The column was washed with 50 mL binding buffer and eluted with a pH 3-7 gradient elution buffer (0.1 M sodium citrate, pH 3.0). Fusion proteins were dialyzed in 1 x phosphate-buffered saline buffer (137 mM NaCl, 2.7 mM KCl, 10 mM Na₂HPO₄, 1.8 mM KH₂PO₄, pH 7.4) using a Slide-A-Lyzer Dialysis Cassette (Thermo Fisher Scientific). For measurements of hNRG4-Fc plasma half-life, male C57BL/6J mice received an i.p. injection of hNRG4-Fc (2 mg/kg body weight). NRG4-Fc fusion protein was quantified using an ELISA kit for human IgG1 Fc (Bethyl Laboratories Inc.).

Sandwich ELISA for hNRG4-Fc measurement Goat anti-human polyvalent immunoglobulins antibody (Sigma, 11761) were diluted in 100 mM sodium carbonate, pH 9.6 buffer at 1:1000 ratio and coated on an ELISA plate at 4° C. overnight. The plate was washed with wash buffer (PBS +0.5% triton-x 100) three times. After blocking with 5% BSA for 1 hour at room temperature, 100 ul of diluted samples or human Fc standard (Thermo Fisher, 10702-HNAH-5) were added into each well and incubated for 2 hours at room temperature. The plate was washed three times with wash buffer, and diluted HRP-conjugated goat anti-human IgG (Fc specific) antibody (Sigma, A0170) in 5% BSA was added. After 1 hour TMB reagent (Thermo Fisher, PI-34028) was used to develop the plate following manufacture's instruction.

Immunoblotting analysis For total lysates, livers were homogenized in a lysis buffer containing 50 mM Tris (pH 7.5), 150 mM NaCl, 5 mM NaF, 25 mM β-glycerol phosphate, 1 mM sodium orthovanadate, 10% glycerol, 1% Triton X-100, 1 mM dithiothreitol (DTT), and freshly added protease inhibitors (Roche). Cells were harvested and homogenized with lysis buffer containing 2% sodium dodecyl sulfate (SDS), 50 mM Tris-HCl (pH 6.8), 10 mM DTT, 10% glycerol, 0.002% bromphenol blue, and freshly added protease inhibitors. Immunoblotting experiments were performed using specific antibodies. For immunoblots, antibodies phospho-ErbB4 (Y1284) (catalog 4757), total ErbB4 (catalog 4795) were from Cell Signaling Technology. Antibody against Hsp90 (Sc-13119) and tubulin (Sc-32293) were from Santa Cruz. Antibodies against Gpnmb (AF2330) was from R&D. Antibody against Fc (A10648), MHC-II (14-5321-85) were from Invitrogen. Trem2 antibody was a generous gift from Dr. Marco Colonna (Washington University in St. Louis).

Data analysis Processing of the sc-RNAseq dataset for chow and NASH NPCs (GSE129516) was previously described (Xiong et al., 2019 Mol Cell 75, 644-660 e645). For Nrg4 WT and KO NPC scRNA-seq dataset, gene expression matrix for each sc-RNAseq sample was generated by CellRanger. Pipeline (10x Genomics) and raw data were processed further with Seurat package (version 3.1.2). Specifically, genes expressed by less than 3 cells were excluded from further analysis. Cells with fewer than 200 detectable genes, greater than 8,750 genes or greater than 20% mitochondrial genes were excluded from analysis. After removing potential doublets, a final dataset with 19,567 genes measured on 33,044 cells were used for downstream analysis.

For both datasets, filtered gene expression counts for each cell were log-normalized with a scale factor of 10,000. To remove potential batch effects, top 2,000 informative genes were selected for each sample through the variance stabilizing transformation and used for the further data integration through the canonical correlation analysis (CCA).

Clustering and cell typing After aligning the top 30 dimensions according to CCA, principal component analysis (PCA) was performed on the integrated data and the first 100 PCs were extracted. An elbow algorithm was used to find the optimal number of PCs to construct Uniform Manifold Approximation and Projection (UMAP) plots. Cells were clustered using the unsupervised learning algorithm with a resolution of 0.1. The cluster marker genes were collected using the Wilcoxon rank-sum test between the cells in a single cluster and all other cells with log fold change threshold of 0.25. The identity for each cluster was further assigned based on the prior knowledge of marker genes. Sub-clustering analysis were performed at higher resolutions for the major cell types, including T cells, macrophages, and dendritic cells.

Data visualization To generate virtual flow, the normalized UMI count matrix was adjusted by adding a normally distributed noise with a mean of 1 and standard deviation of 0.1. The plots were generated by ggplot2. The gradient colors were scaled by estimated count. Feature plots, dot plots, violin plots, and heatmaps were generated by Seurat, pheatmap or ggplot2 in R. The differential expressed genes in volcano plots were determined by Wilcoxon ranksum test between the cells in different genotypes or diet conditions within one cell cluster.

RNA velocity analysis RNA velocity analysis was performed using the scVelo (0.2.2) implemented in python. Bam files were first converted to loom files using the velocyto based on the mm10 annotation files. Cells identified as macrophage (n=7,526) were extracted from the loom files for all samples and used for the velocity analysis. The extracted data were filtered and normalized with default parameters. After the moments (first and second) calculation, the velocities were obtained by modeling the transcriptional dynamics of splicing kinetics stochastically. The UMAP coordinates from the Seurat analysis were used as basis for the visualization.

CellPhoneDB analysis CellPhoneDB (v3) was used to analyze intercellular communication between CD8+ T cells and macrophages for chow and NASH samples using the version 2.0.0 of the database (1396 interactions), with default parameters (10% of cells expressing the ligand/receptor). A permutation test (10,000 permutations) was applied to determine statistical significance. Interactions with P<0.05 were considered significant. Due to the lack of intercellular interaction database for mice in CellPhoneDB, the mouse ligand-receptor interaction gene list was generated based on human orthologs utilizing the R package biomaRt.

### Example 1

### Induction of Liver Macrophages Reminiscent of Tumor-Associated Macrophages During Diet-Induced NASH

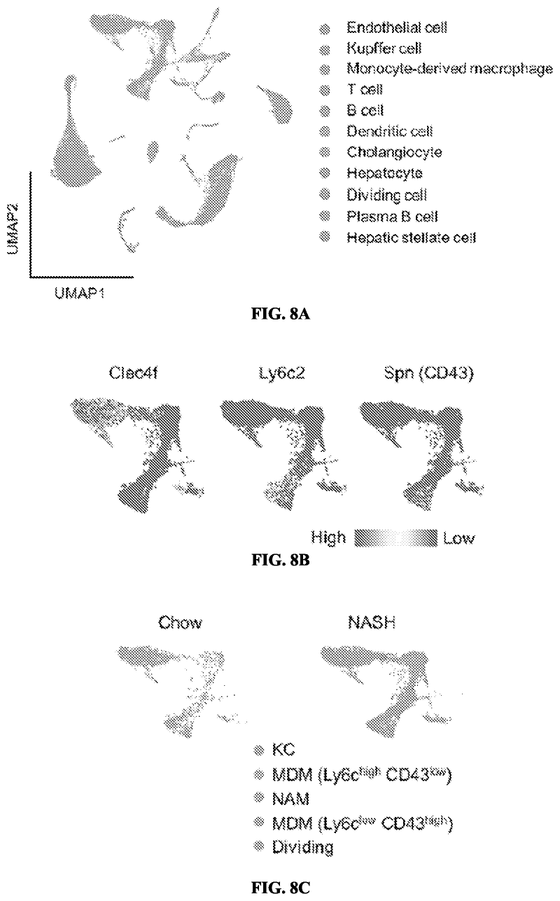

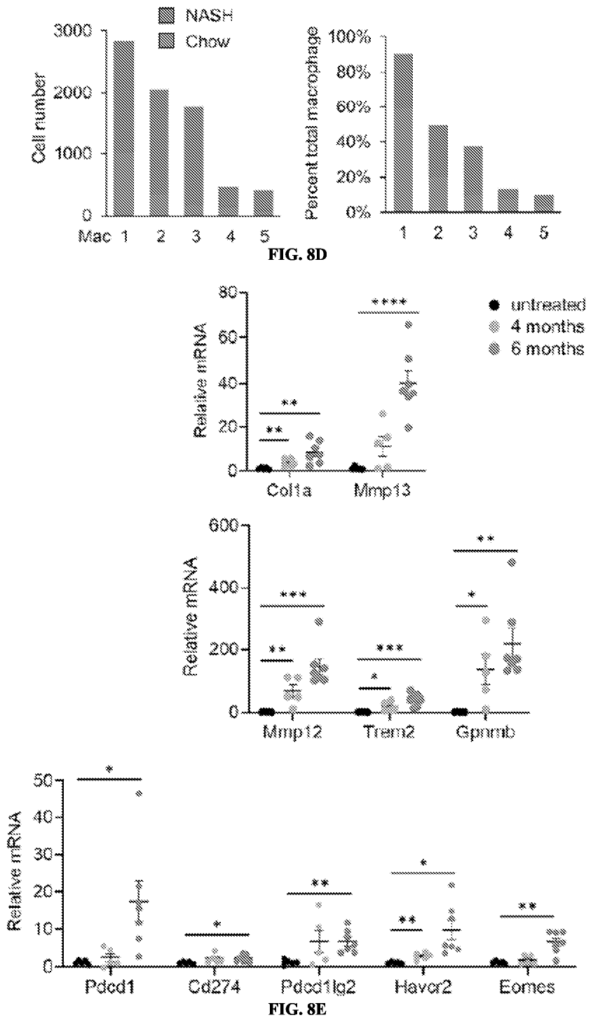

NASH pathogenesis entails metabolic derangements and injury of hepatocytes that trigger a cascade of response by non-parenchymal cells in the liver to restore tissue homeostasis. To test if NASH pathogenesis might induce reprogramming of intrahepatic immune cell populations to facilitate the development of NASH-associated HCC this, the transcriptomic states of macrophages and T cells in healthy and NASH livers was investigated by analyzing a liver NPC sc-RNAseq dataset (Xiong et al., 2019a). UMAP dimensionality reduction analysis identified 11 clusters, corresponding to major cell types in mammalian liver, including endothelial cell, macrophage, T cell, B cell, dendritic cell, cholangiocyte, hepatocyte, and hepatic stellate cell (FIG. 8A).

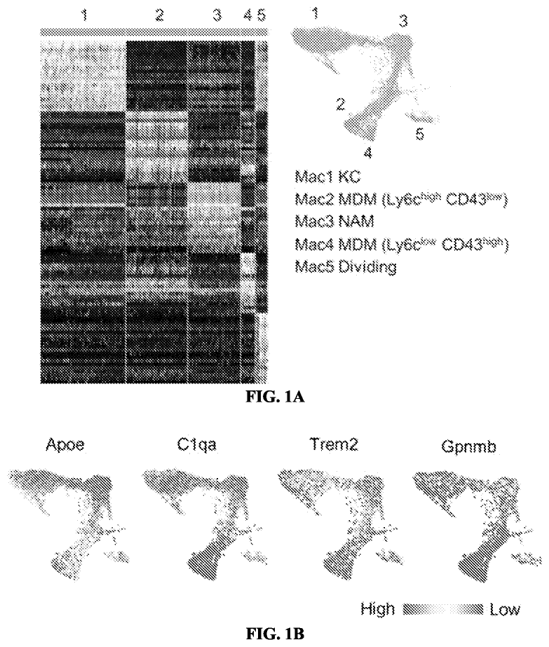

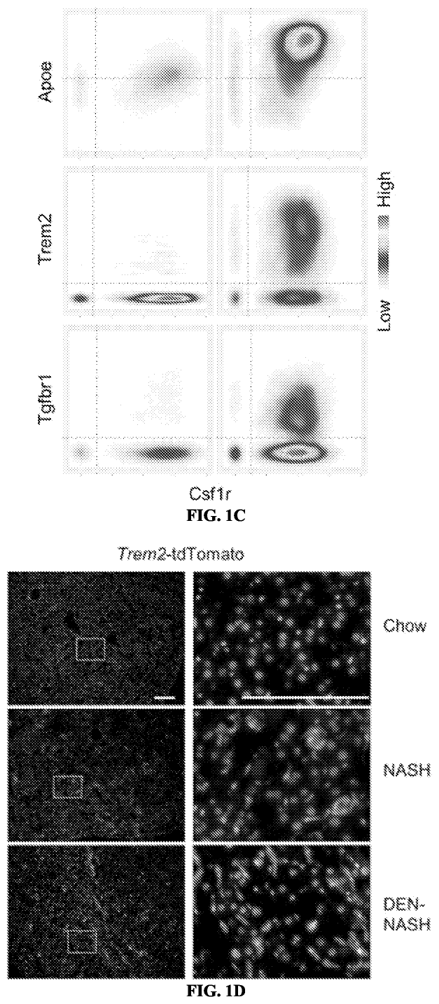

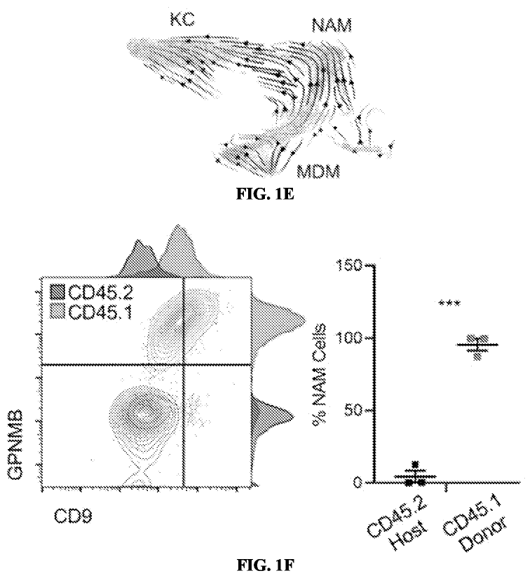

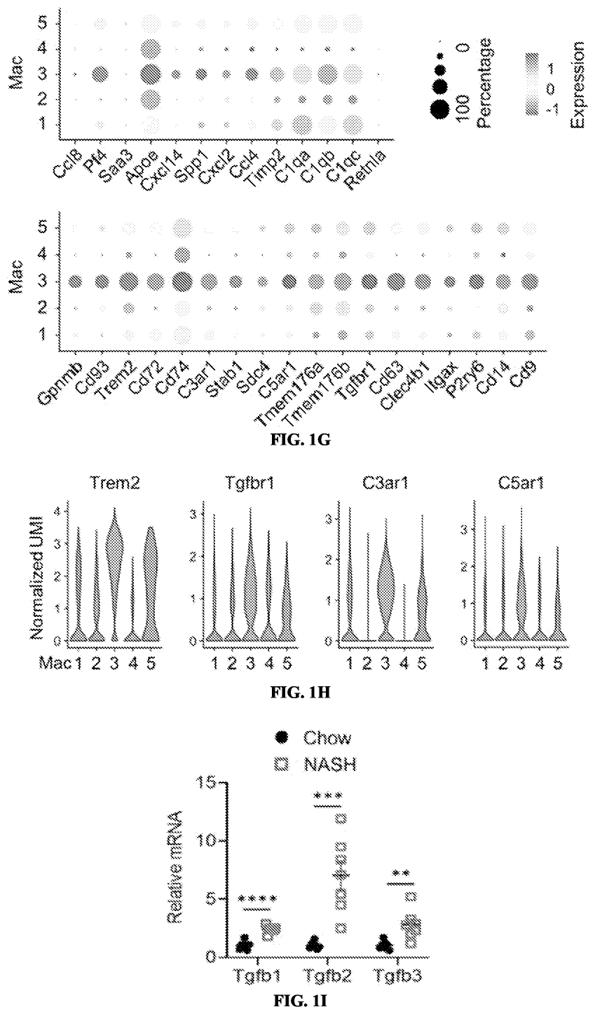

The macrophage cluster contained a total of 7,526 cells and represented the largest immune cell population among NPCs in the liver. Subcluster analysis revealed five macrophage subtypes exhibiting distinct transcriptional signatures (Mac1-5, FIG. 1A). Mac1 represents Kupffer cells, the resident macrophage of the liver, whereas Mac2 and Mac4 correspond to classical and non-classical monocytes that can be distinguished by their expression of Ly6c2 and Cd43 (FIG. 8B). Mac3 contains macrophages originated primarily from NASH liver (FIGS. 8C and 8D) and accordingly, they were termed NASH-associated macrophages (NAMs). A notable feature of NAMs is that they express a set of unique molecular markers, including Apoe, C1qa, Trem2, and Gpnmb (FIG. 1B), which have been demonstrated to be enriched in tumor-associated macrophages (TAMs) in skin, liver, lung, breast, bladder, colon, stomach, pancreas, and kidney cancers (Bulla et al., 2016; Molgora et al., 2020; Zhang et al., 2019). To visualize this, virtual flow cytometry was performed on the macrophage cluster by gating for single-cell mRNA expression of Csf1r, a pan-macrophage marker, in combination with NAM markers. Compared to chow control, macrophages exhibiting high expression of Apoe, Trem2, and Tgfbr1 were strongly enriched in the livers from NASH mice (FIG. 1C).

To track the dynamic regulation of Trem2-expressing macrophages in NASH, a knockin mouse strain that expresses Cre recombinase fused to the C-terminus of the endogenous Trem2 via the self-cleavage 2A peptide was generated. Trem2-Cre mice were crossed with a Rosa26-tdTomato reporter strain to label the Trem2 macrophage lineage. A small number of tdTomato-positive macrophages were found in chow-fed mouse liver (FIG. 1D). Following diet-induced NASH, the abundance of tdTomato-positive cells was markedly increased, some of which formed aggregates that resemble crown-like structure observed in adipose tissue during obesity. Whether Trem2-positive macrophages are induced during HCC development was assessed by combining carcinogen treatment and NASH induction. Postnatal day 15 pups were treated with diethylnitrosamine (DEN) and treated mice were subjected to NASH diet feeding to model the development of NASH-HCC. Analysis of hepatic gene expression indicated that mRNA levels of liver fibrosis genes (Co11a1, Mmp13) and NAM markers (Mmp12, Trem2, Gpnmb) progressively increased during tumor induction (FIG. 8E). Consistent with these results, robust induction of tdTomato-positive macrophages was observed in this NASH liver cancer model (FIG. 1D).

### Example 2

### Regulation of NASH-Associated Liver Macrophages

RNA velocity analysis was performed to probe the relationship among different macrophage subtypes. This analysis is based on the relative abundance of unspliced pre-mRNA and mature mRNA to infer the trajectory of cell states. As shown in FIG. 1E, notable cell state transitions from classical monocytes to NAMs and KC subclusters were observed. These observations were consistent with previous findings that monocytes provide a cellular source for newly formed Kupffer cells during chronic liver injury and inflammation (Molawi and Sieweke, 2015; Tacke and Zimmermann, 2014). To define the cellular origin of NAMs in NASH liver, bone marrow transplantation was performed using donor cells expressing CD45.1, a pan-hematopoietic cell surface marker, into host mice harboring a distinct allele (CD45.2). Following 6 weeks of feeding a choline-deficient amino acid-defined HFD diet (CDA-HFD), a diet that robustly induces NASH pathologies in mice, CD45 isotype expression on NAMs was analyzed using flow cytometry. Remarkably, over 95% of NAMs, as marked by CD9 and GPNMB, displayed CD45.1 expression (FIG. 1F), indicating that bone marrow-derived monocytes serve as the primary source of intrahepatic NAMs during NASH.

The nature of the extracellular signals that trigger the induction of Trem2+ macrophages subpopulation remains largely unknown. To address this, differential gene expression analysis for secreted factors and membrane proteins was performed among five macrophage subclusters. This analysis identified a set of putative ligands exhibiting enriched expression in NAMs, including Pf4, Apoe, Spp1, and Cc14 (FIG. 1G). In addition, a group of 17 genes encoding putative membrane receptors that exhibited preferential expression in NAMs were identified. Several of these genes, including Gpnmb, Trem2, Cd74, C3ar1, Stab1, and C5ar1 have been previously shown to mark TAMs in various cancer types. Violin plots indicated that mRNA expression of Trem2, Tgfbr1, C3ar1, and CSar1 exhibited higher expression in NAMs compared to other sub clusters (FIG. 1H). Interestingly, Mac5, which represents proliferating cells, also displayed elevated expression of these genes, suggesting that macrophages with characteristics of NAMs likely undergo active proliferation in the liver. qPCR analysis revealed that hepatic mRNA expression of all three TGFβ ligands was elevated upon diet-induced NASH in mice (FIG. 1I). Analysis of the sc-RNAseq dataset indicated that Tgfb1 is expressed by diverse cell types in the liver, whereas Tgfb2 and Tgfb3 exhibited more restricted expression to cholangiocytes and HSCs (FIG. 8F), suggesting that autocrine and paracrine TGFB signaling may contribute to NAM induction in the liver. To examine whether TGFβ promotes NAM gene expression in a cell-autonomous manner, treatments on bone marrow-derived macrophages (BMDM) were performed and TGFβ robustly stimulated mRNA expression of Trem2, Gpnmb, Apoe, and Tgfbr1 in cultured macrophages (FIG. 1J). TGFβ signaling may be common extracellular cue that promotes NAM induction in NASH liver and TAMs during tumorigenesis

### Example 3

### NASH Pathogenesis Triggers CD8+ T Cell Exhaustion in the Liver

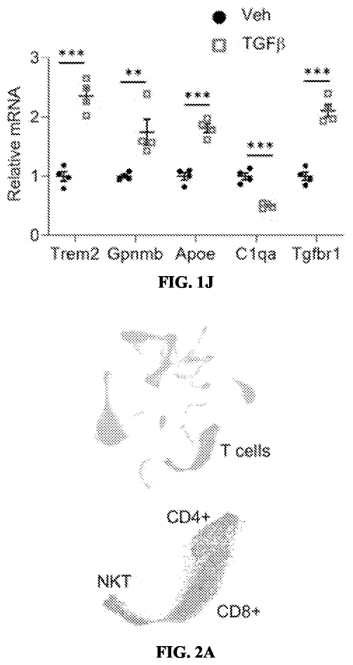

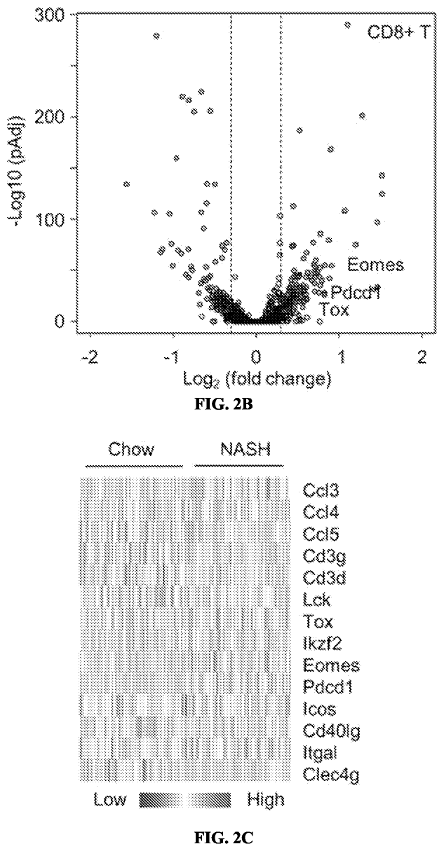

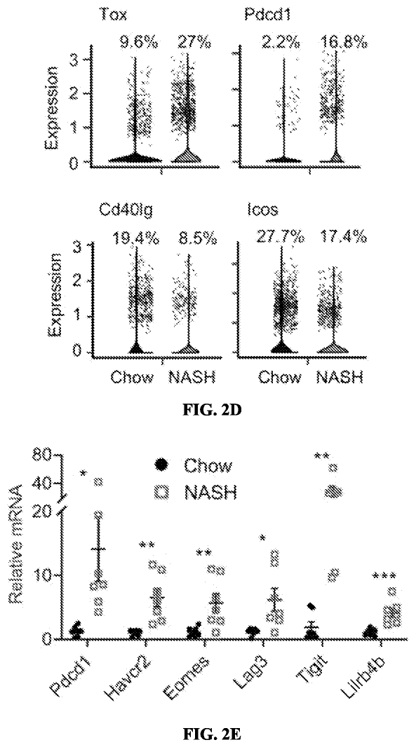

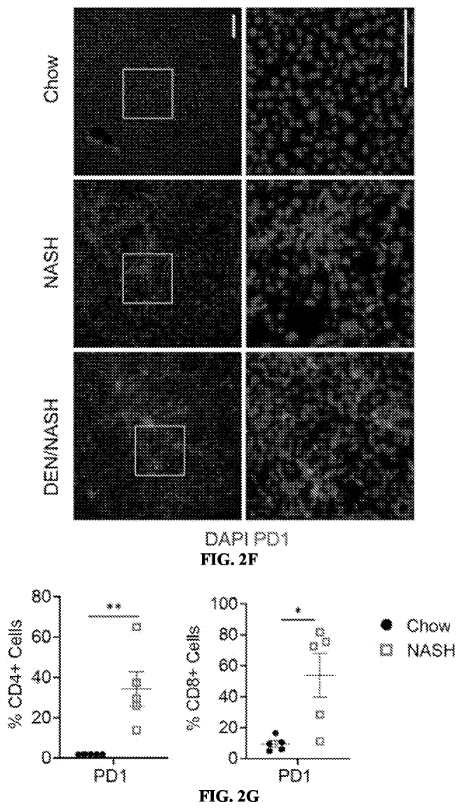

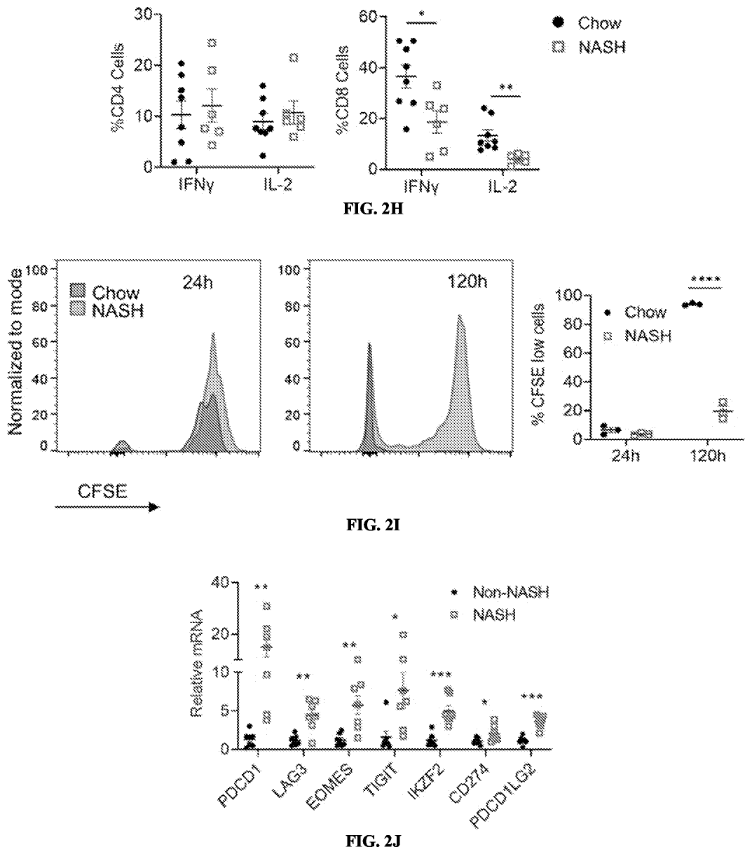

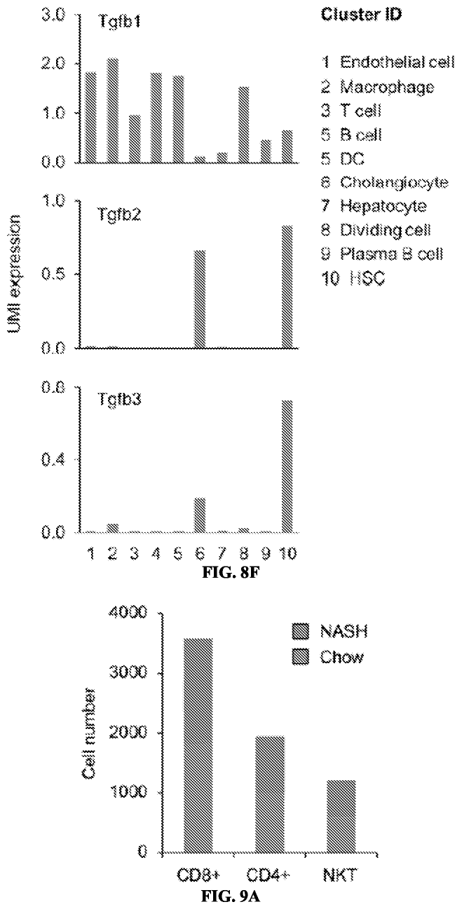

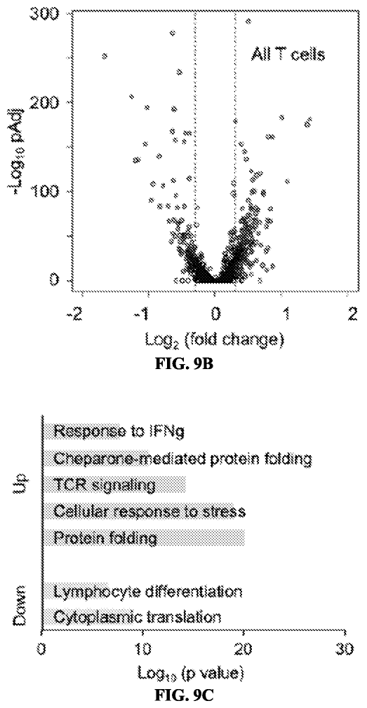

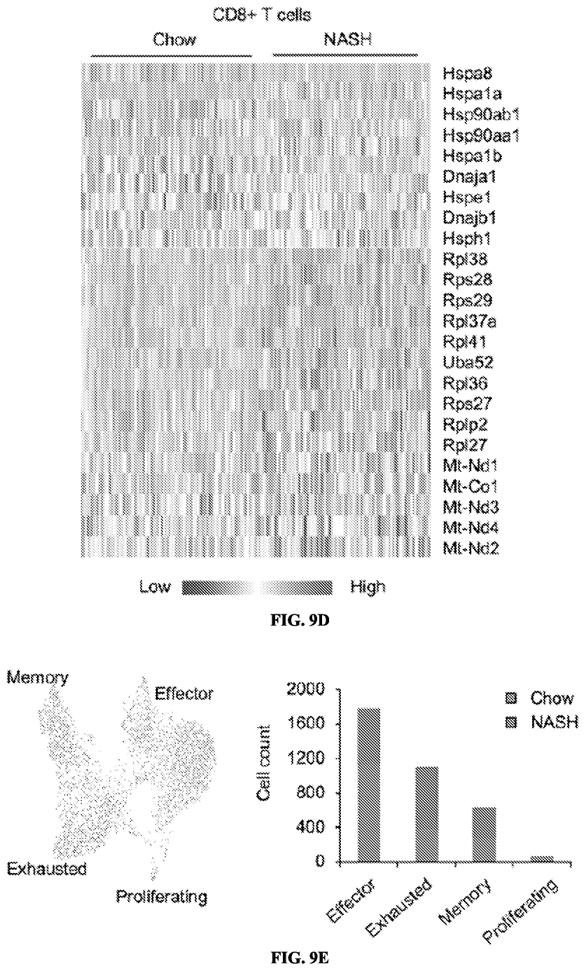

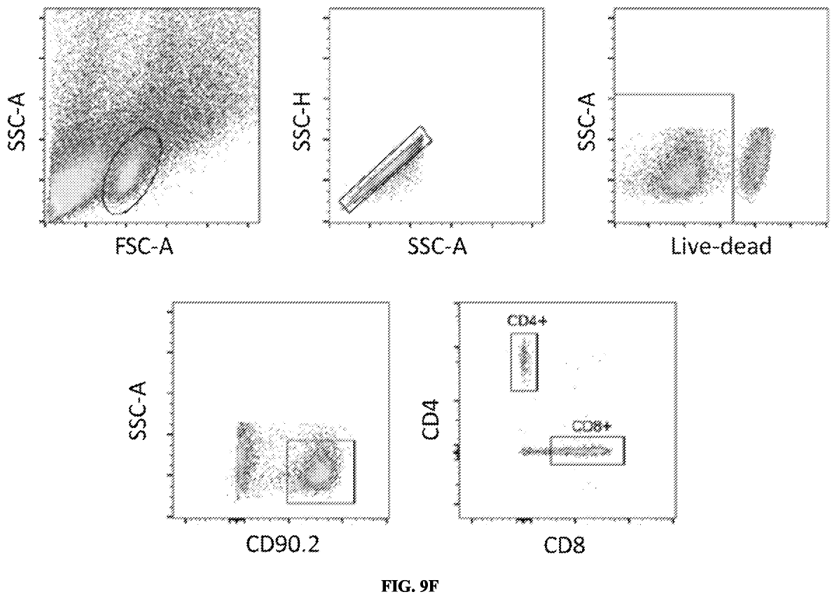

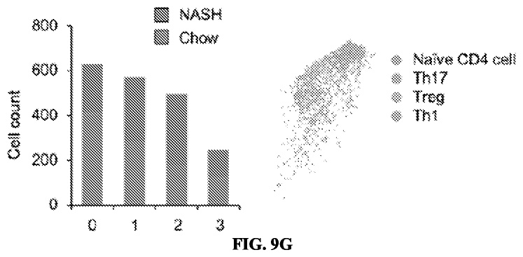

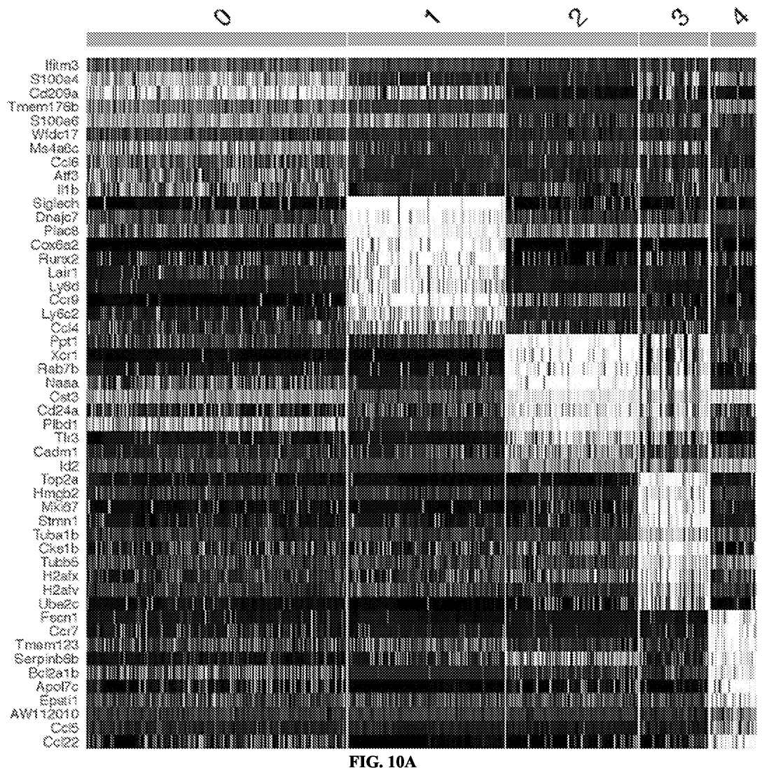

Unlike macrophages, which undergo massive expansion during diet-induced NASH, the total number of T cells remained comparable between chow and NASH groups (FIG. 9A). Based on their transcriptomic signature, T cells can be further divided into three subclusters that represent intrahepatic CD8+, CD4+, and Natural Killer T (NKT) cells (FIG. 2A). Differential gene expression analysis on CD8+subcluster revealed a set of genes dysregulated in NASH (FIG. 2B). Pathway analysis indicated that the upregulated genes were enriched for chaperone-mediated protein folding, cellular stress response, and TCR and IFNγ signaling, whereas the downregulated genes corresponded to cytoplasmic protein translation and lymphocyte differentiation (FIG. 9B). mRNA expression of several genes involved in TCR signaling (Cd3d, Cd3g, Lck) and cytokine response (Cc13, Cc14, Cc15) were increased in CD8+ T cells from NASH mice, likely reflecting persistent stimulation of T cell signaling in the context of liver injury and inflammatory response (FIG. 2C). On the contrary, mRNA levels of several T cell stimulatory factors (Icos, Cd401g, Itga1, Clec4g) were decreased following diet-induced NASH. mRNA expression of many genes involved in protein folding and stress response was elevated in NASH CD8+ T cells, whereas those involved in ribosome biogenesis and mitochondrial oxidative metabolism were suppressed (FIGS. 9C and 9D). These results illustrated that intrahepatic CD8+ T cells undergo marked transcriptomic reprogramming following NASH induction.

Among genes upregulated in NASH liver, mRNA expression increased for several genes known to be involved in CD8+ T cell exhaustion, including Pdcd1, Tox, and Eomes (FIG. 2C). Impaired effector function and cytokine release by cytotoxic T cells result from excess inhibitory receptor signaling in CD8+ T cells and contribute to defective cancer immunity. Single-cell gene expression analysis revealed that the frequency of CD8+ cells exhibiting high Pdcd1 expression increased from 2.2% to 16.8% (FIG. 2D). The frequency of CD8+ T cells with high Tox expression also increased from 9.6% to 27%. In contrast, fewer CD8+ T cells exhibited high expression of Cd401g and Icos, costimulatory receptors involved in T cell activation. Subcluster analysis of CD8+ T cells revealed four subpopulations that correspond to effector, exhausted, memory, and proliferating T cells (FIG. 9E). Exhausted CD8+ T cells were more prevalent in NASH than healthy liver. In contrast, the effector T cell subpopulation was diminished in NASH liver. CD8+T cells acquire the molecular characteristics of exhaustion following NASH induction.

Hepatic gene expression analysis indicated that mRNA levels of Pdcd1, Havcr2, Eomes, Lag3, Tigit, and Li1rb4b were strongly increased following diet-induced NASH (FIG. 2E). Similarly, mRNA expression of this set of genes was elevated during NASH-HCC development (FIG. 8E). anti-PD1 (encoded by Pdcd1) immunofluorescence staining and flow cytometry was performed to assess T cell exhaustion in NASH liver. While few PD1-positive T cells were observed in the liver from chow-fed mice, they were readily detectable on liver sections from mice subjected to NASH and DEN/NASH treatments (FIG. 2F). Flow cytometry analysis indicated that, compared to chow, PD1-high T cells among the CD8+ population were increased from 9.6% to 54.0% in the NASH group (FIGS. 2G and 9F). Interestingly, CD4+ T cells also showed higher PD1 expression in NASH liver. Consistent with these molecular features of T cell exhaustion, CD8+, but not CD4+, T cells from NASH livers exhibited reduced IFNγ and IL-2 secretion following stimulation with PMA/ionomycin (FIG. 2H). Further, proliferation of liver CD8+ T cells in response to CD3/CD28 Dynabeads activation was greatly diminished by NASH, compared to chow control (FIG. 2I). To assess the relevance of these findings in human NASH, gene expression analysis was performed on liver biopsies from a cohort of individuals without or with NASH. As shown in FIG. 2J, mRNA levels of PDCD1, LAG3, EOMES, TIGIT, IKZF2, CD274, and PDCD1LG2 were strongly induced in human NASH livers.

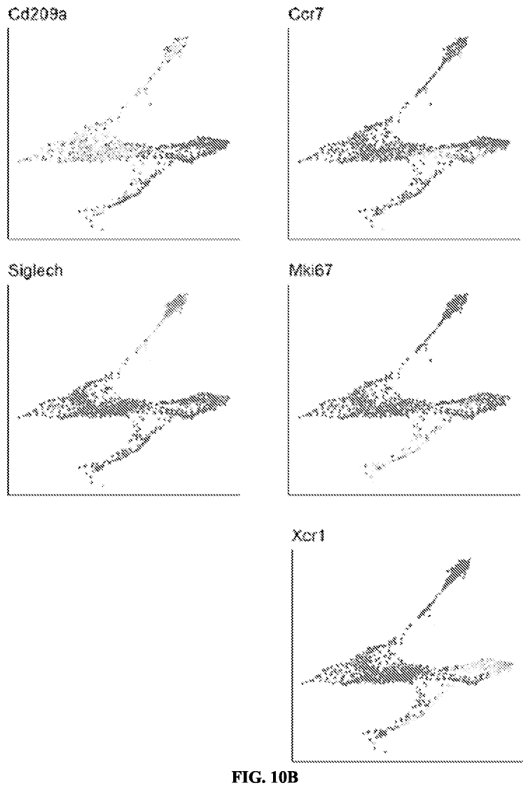

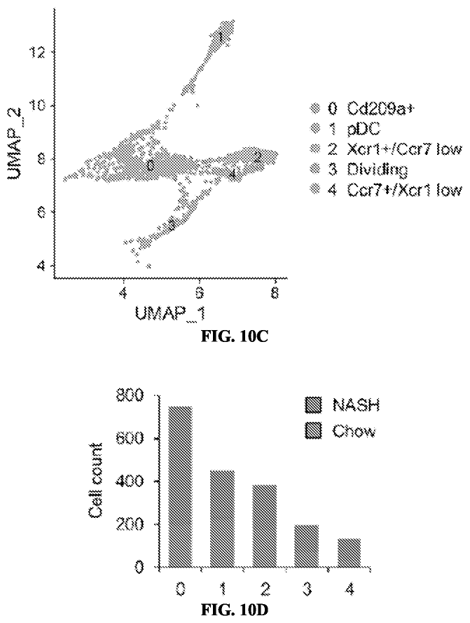

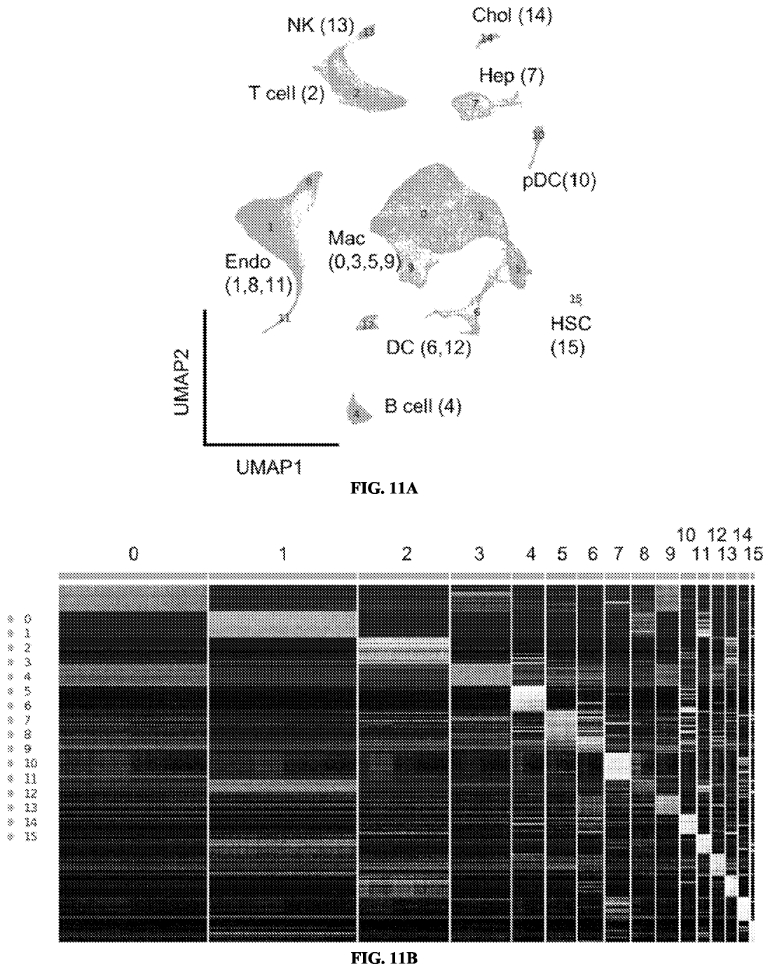

Subclustering analysis of CD4+ T cells identified four subtypes that correspond to Naïve CD4 T cells, regulatory T cells (Tregs), Th17, and Th1 cells (FIG. 9G). The abundance of these subtypes in the liver appeared comparable between healthy and NASH mice. Dendritic cells (DCs) play an important role in antigen presentation and adaptive immune response. Five DC subpopulations were identified based on their marker gene expression: Cd209+, plasmacytoid DC, Xcr1^{high} Ccr7^{low}, Xcr1^{low} Ccr7^{high}, and dividing DCs (FIGS. 10A-10C). Interestingly, Cd209+ DCs appeared to undergo pronounced expansion in NASH liver (FIG. 10D). Taken together, the results illustrated profound reprogramming of the liver immune microenvironment during NASH pathogenesis. The induction of TAM-like macrophages and exhausted CD8+ T cells may predispose NASH mice to the development of liver cancer.

### Example 4

### NRG4 Shapes the Liver Microenvironment and Serves as a Checkpoint for NASH-Associated HCC

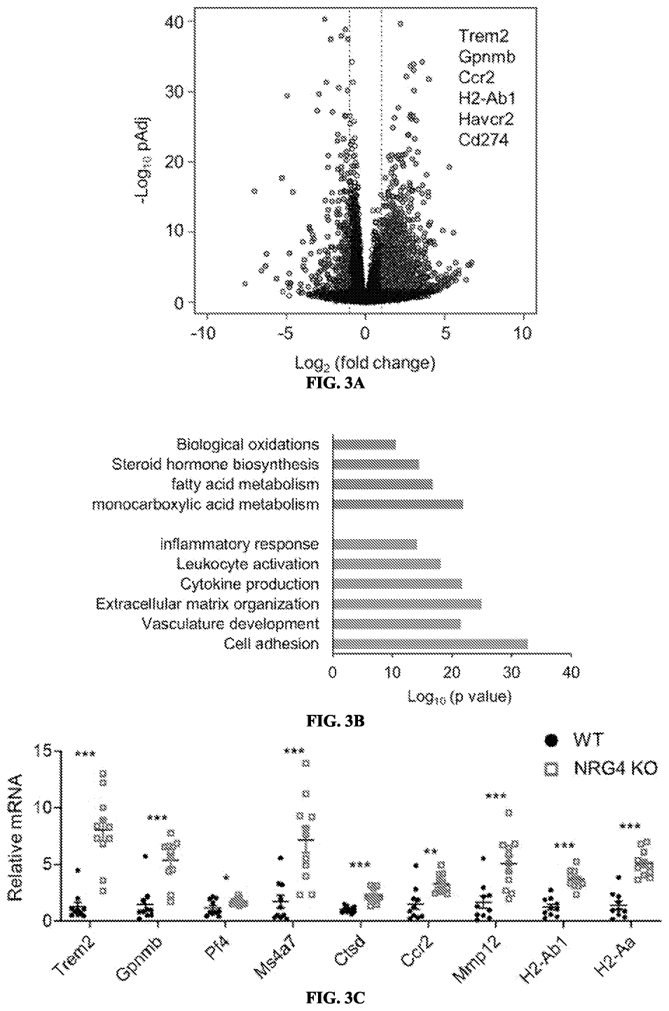

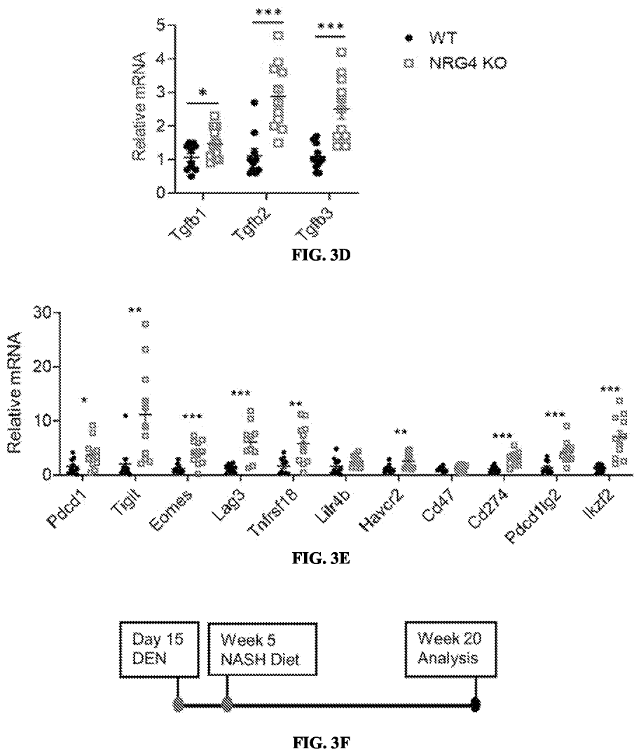

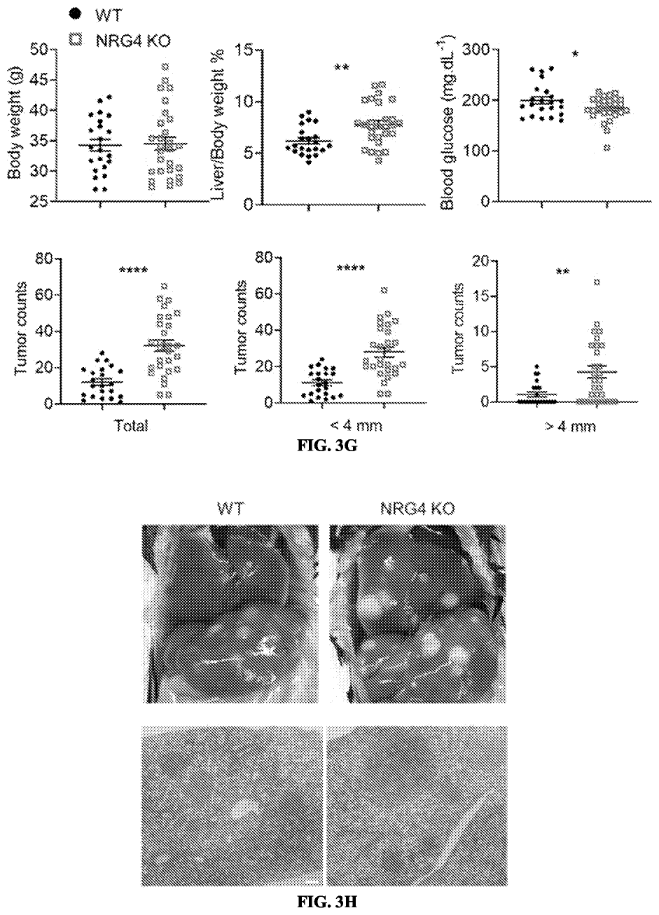

Bulk RNA sequencing was performed on liver RNA isolated from NRG4 knockout (KO) mice and wild type (WT) littermate control following six months of NASH diet feeding. Differential gene expression analysis revealed 1,290 and 272 genes exhibiting more than 2-fold changes in mRNA expression (FIG. 3A). Gene ontology analysis indicated that genes downregulated in NRG4 KO livers were enriched for pathways involved in substrate oxidation, lipid metabolism, and steroid hormone biosynthesis (FIG. 3B), whereas upregulated genes were enriched for immune cell adhesion, leukocyte activation, and inflammatory response. Among the upregulated genes are NAM-enriched genes, including Trem2, Gpnmb, Pf4, Ms4a7, Ctsd, Ccr2, Mmp12, H2-Ab1, H2-Aa, and the TGFB family of ligands (FIGS. 3C-3D). Remarkably, NRG4 deficiency exacerbated NASH-associated induction of genes involved in T cell exhaustion, such as Pdcd1, Tigit, Eomes, and Lag3. In addition, mRNA expression of Cd274, which encodes PD-L1, a PD-1 ligand, and Pdcd11g2, which encodes PD-L2, also showed increased mRNA expression in the livers from NRG4 KO mice (FIG. 3E).

HCC in NRG4 KO mice and WT littermates were induced using the DEN/NASH protocol (FIG. 3F). Under this condition, WT and KO mice gained comparable body weight during the study period (FIG. 3G). Tumor nodules of varying sizes were observed in both genotypes (FIG. 3H). Compared to control, NRG4 KO mice exhibited significantly increased tumor incidents. Total tumor count was 2.6-fold higher in NRG4 KO group than WT control. Both tumors of smaller sizes (<4 mm) and larger sizes (>4 mm) were increased in the KO group. As a result of increased tumor burden, liver weight to body weight ratio was significantly higher in NRG4 KO group. Blood glucose levels were slightly lower in the KO cohort, likely due to impairments of hepatic metabolic functions. As such, NRG4 inactivation exacerbated the dysregulations of the liver immune microenvironment and rendered mice more prone to development of NASH-associated HCC.

### Example 5

### NRG4 Restrains Tumor-Prone Liver Immune Microenvironment During NASH

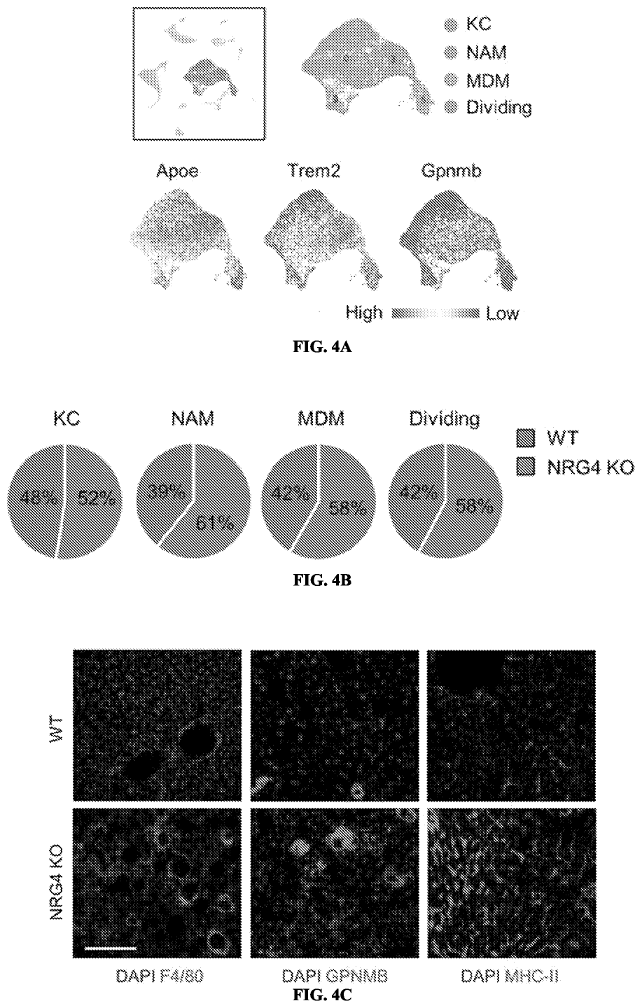

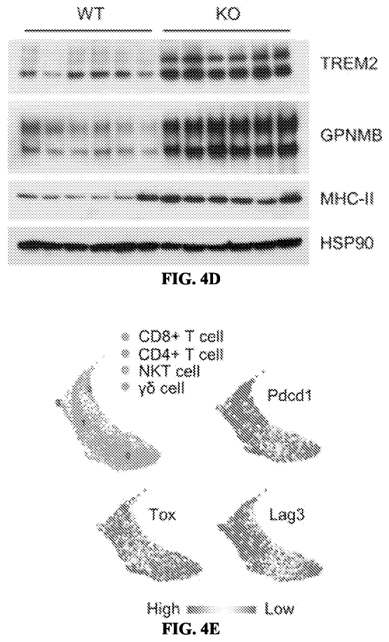

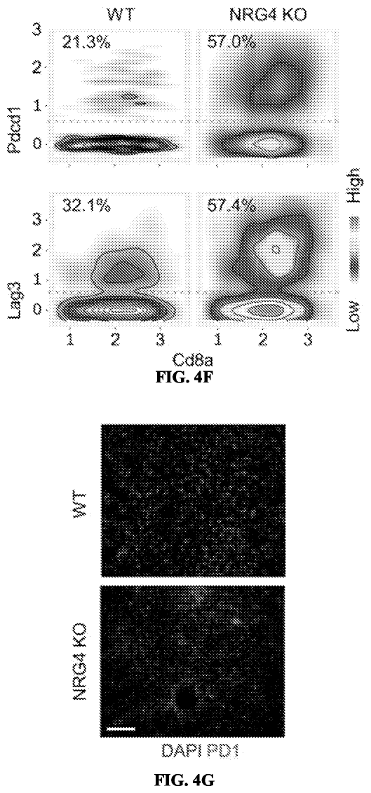

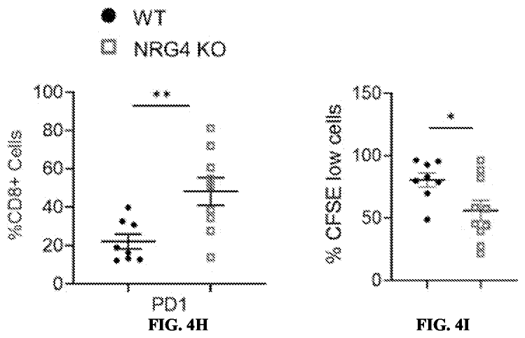

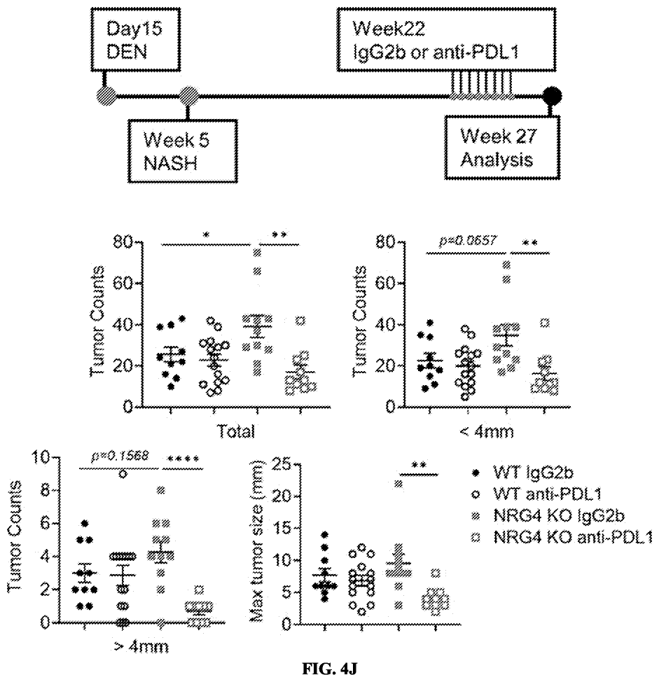

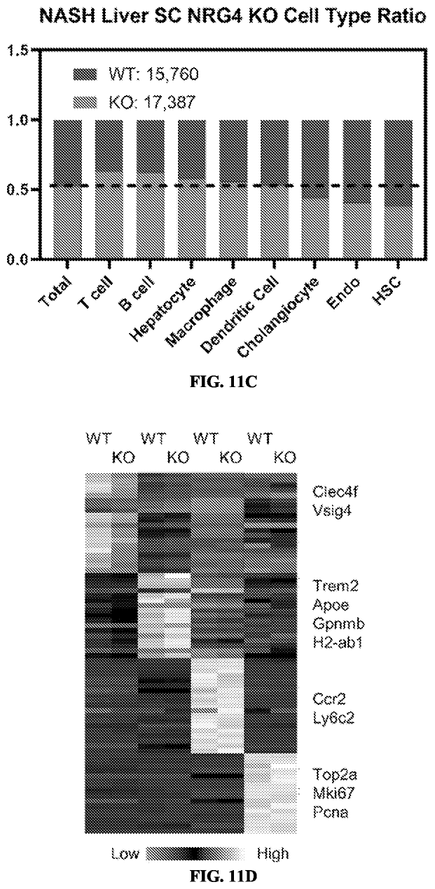

sc-RNAseq was performed on NPCs isolated from WT and NRG4 KO mouse livers following NASH diet feeding. UMAP analysis revealed a total of 16 clusters that represent eight major liver cell types (endothelial, macrophage, T, B, DC, hepatocyte, cholangiocyte, HSC) expressing unique molecular markers (FIG. 11A-11C). The macrophage cluster contains a total of 12,824 cells and represents the largest population among the NPCs. Subcluster analysis revealed four macrophage subtypes: KC, MDM, NAM, and dividing cells (FIGS. 4A and 11D). NAMs displayed prominent expression of Apoe, Trem2, and Gpnmb. While cell counts for the resident macrophage population (KC) remained comparable between two genotypes, a notable expansion of Trem2+ NAMs was observed in NRG4 KO livers, which accounted for approximately 61% of the NAM population (FIG. 4B). The MDM and dividing macrophage subclusters also showed expansion in the livers from NRG4 KO mice. Accordingly, immunofluorescence indicated that F4/80, GPNMB, and MHC-II were markedly increased in NRG4 KO livers (FIG. 4C). A subset of GPNMB-positive macrophages form “crown-like structures” similar to those observed in adipose tissue inflammation during obesity. Immunoblotting studies showed that protein levels of TREM2, GPNMB, and MHC-II were elevated in NRG4 KO livers (FIG. 4D). These findings demonstrated that NRG4 deficiency promoted the expansion of monocyte-derived TAM-like macrophages in the liver.

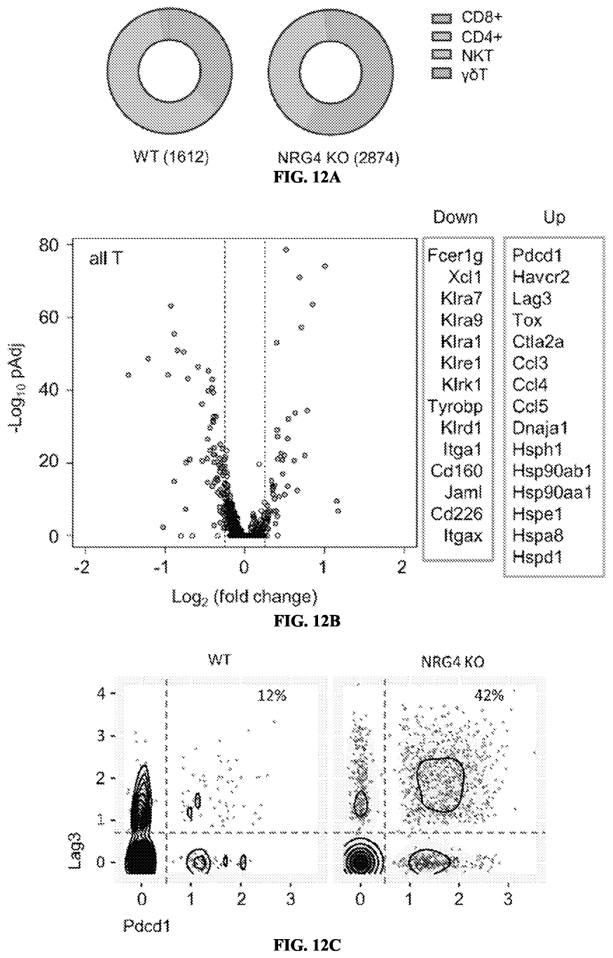

Subcluster analysis of T cells revealed four subtypes: CD4+, CD8+, NKT, and a small group that represents γδT cells (FIGS. 4E and 12A). Total number of T cells and the CD8+ population showed a notable expansion in NRG4 KO livers. Differential gene expression analysis revealed a group of genes with altered expression in NRG4 KO T cells, as visualized by volcano plot (FIG. 12B). The upregulated genes include those involved CD8+ T cell exhaustion, such as Pdcd1, Havcr2, Lag3, and Tox. Interestingly, mRNA expression for genes involved in chaperone-mediated protein folding and stress response (Dnaja1, Hsph1, Hsp90aa1, Hsp90ab1, Hspe1, Hspe8) and chemokine signaling (Cc13, Cc14, Cc15) were also increased. Many down-regulated genes correspond to NKT cell functions, including Xc11 and members of the Killer cell lectin-like receptor family. Analysis of single-cell gene expression for these markers by virtual flow revealed increased frequency of CD8+ T cells that harbor high levels of expression for Pdcd1 and Lag3 (FIG. 4F), indicating that NRG4 deficiency promotes T cell exhaustion in the liver. Pdcd1 and Lag3 double positive CD8+ T cells increased from 12% in WT livers to approximately 42% in NRG4 KO livers (FIG. 12C). NRG4 deficiency likely exacerbates pathological reprogramming of T cell transcriptome under metabolic stress conditions.

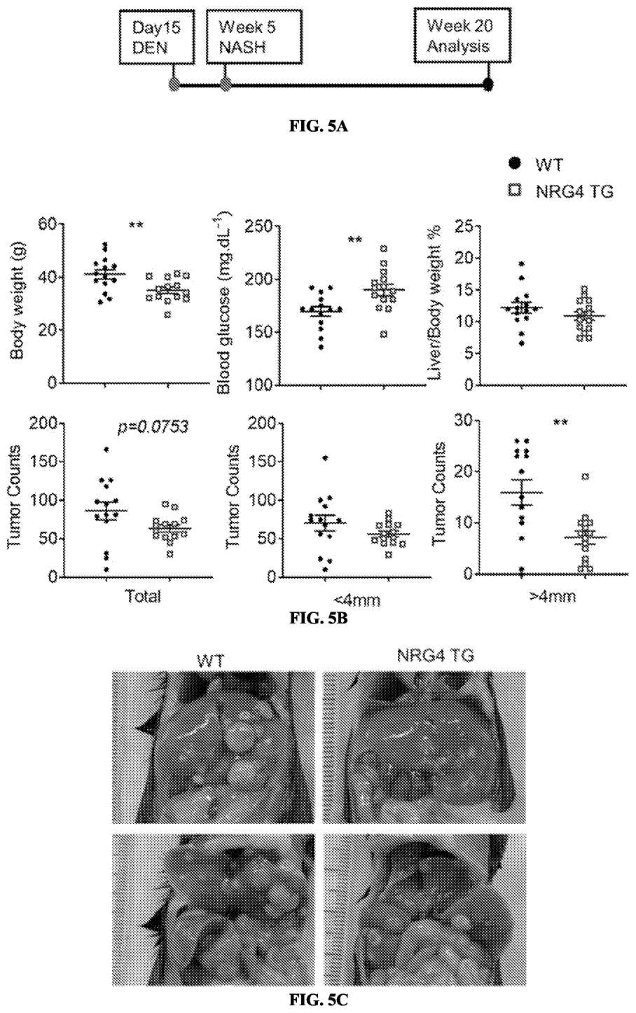

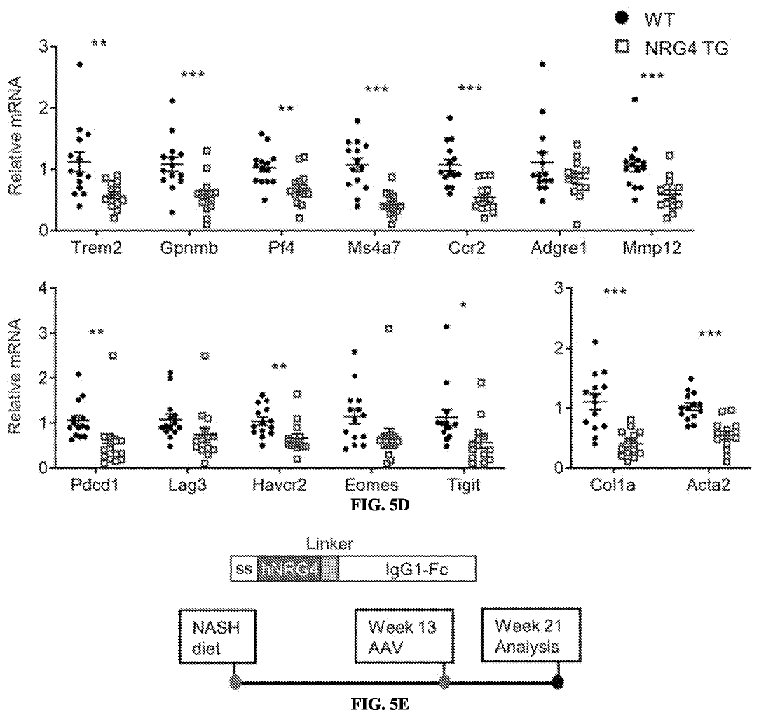

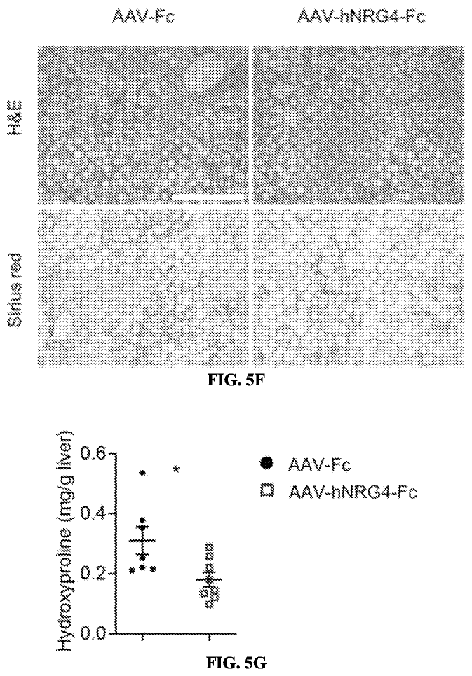

The exacerbation of T cell exhaustion in mice lacking NRG4 was confirmed by anti-PD-1 immunofluorescence staining and flow cytometry analysis (FIGS. 4G-4H). As early as three months into NASH diet, PD-1 positive CD8+ T cells increased from 22.1% in WT to 48.2% in NRG4 KO mice. CD8+ T cells isolated from NRG4 KO mouse livers exhibited reduced proliferative response upon activation by CD3/CD28 Dynabeads (FIG. 5I). These findings suggested that T cell dysfunction may contribute to increased liver tumorigenesis in NRG4 deficiency. Treatments were performed on WT and NRG4 KO mice subjected to the DEN/NASH tumor protocol with a blocking antibody against PDL1 (anti-PDL1) or IgG2b isotype control (FIG. 5J). Anti-PDL1 elicited modest effects on liver tumorigenesis in WT mice. In contrast, PD-L1 blockade significantly reduced tumor counts and maximal tumor size in NRG4 KO livers, suggesting that impaired checkpoint function contributes to increased NASH-HCC incidence in NRG4 KO mice.

### Example 6

### Transgenic Elevation of NRG4 Protects Mice From NASH-Associated Liver Cancer

To assess whether transgenic overexpression of NRG4 restores the liver immune microenvironment and restricts the development of NASH-associated HCC, cohorts of NRG4 transgenic mice and WT littermates were subjected to the DEN/NASH liver cancer protocol, as described above, and analyzed liver tumorigenesis following 20 weeks of NASH diet feeding (FIG. 5A). Compared to control, NRG4 TG mice gained slightly less body weight (FIGS. 5B and 5C). In contrast to NRG4 deficiency, transgenic overexpression of NRG4 reduced total liver tumor count. While tumors smaller than 4 mm were comparable between two groups, the average number of tumors larger than 4 mm was significantly reduced in Tg group. Blood glucose levels in the transgenic cohort were higher than control, likely reflecting lower tumor load and improved hepatic function.

Gene expression analysis indicated that mRNA expression of NAM markers (Trem2, Gpnmb) and several other macrophage genes enriched in NAMs (Pf4, Ms4a7, Ccr2, Mmp12) was significantly lower in TG livers (FIG. 5D). Likewise, mRNA expression of genes involved in T cell exhaustion was attenuated by transgenic NRG4, including Pdcd1, Lag3, Havcr2, Eomes, and Tigit. Further, markers for activated HSC and liver fibrosis, including Co11a1 and Acta2, also showed reduced expression in the liver from NRG4 TG mice. Together, the data support a crucial role of NRG4 in modulating the liver immune microenvironment and development of NASH-associated HCC.

### Example 7

### Therapeutic Targeting of NASH-Associated Liver Cancer by NRG4-Fc Fusion Protein

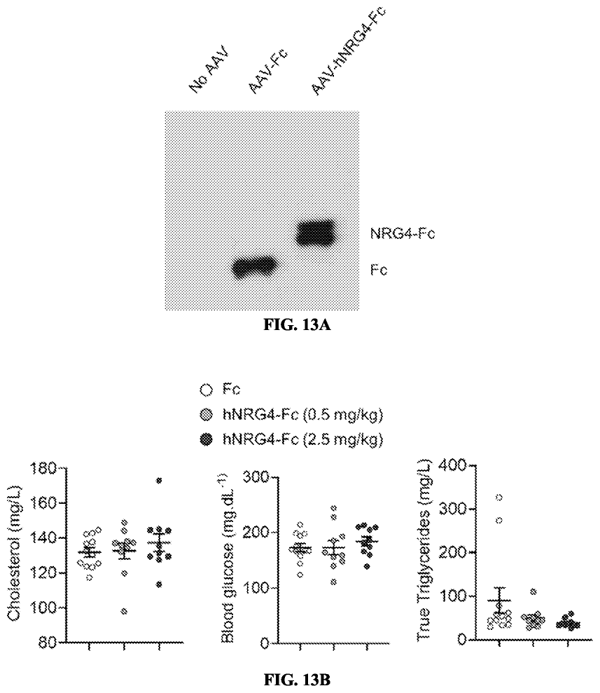

A recombinant adenovirus-associated virus (AAV) vector expressing a secreted fusion protein between amino acids 1-55 of human NRG4 (hNRG4) and the Fc domain of IgG1 was constructed (hNRG4-Fc, FIG. 5E). These two domains were separated by a glycine-serine (GGGGGS; SEQ ID NO: 3) linker that provides additional spatial flexibility for NRG4 to engage its receptor. To examine whether AAV-mediated overexpression of hNRG4-Fc fusion protein elicits protective effects on NASH, WT mice fed NASH diet were transduced with AAV-Fc or AAV-hNRG4-Fc and NASH diet feeding was continued for additional 8 weeks. Robust secretion of Fc and hNRG4-Fc fusion protein was detected in plasma from transduced mice (FIG. 13A). While hepatic steatosis was comparable between two groups, Sirius red staining and measurements of hydroxyproline content revealed that mice transduced with AAV-hNRG4-Fc exhibited less severe liver fibrosis (FIGS. 5F and 5G). Importantly, mRNA expression of NAM-associated genes (Trem2, Gpnmb, Ms4a7, Mmp12) and T cell exhaustion genes (Pdcd1, Havcr2, Lag3, Tigit) was significantly attenuated in response to hNRG4-Fc during NASH progression (FIG. 5H).

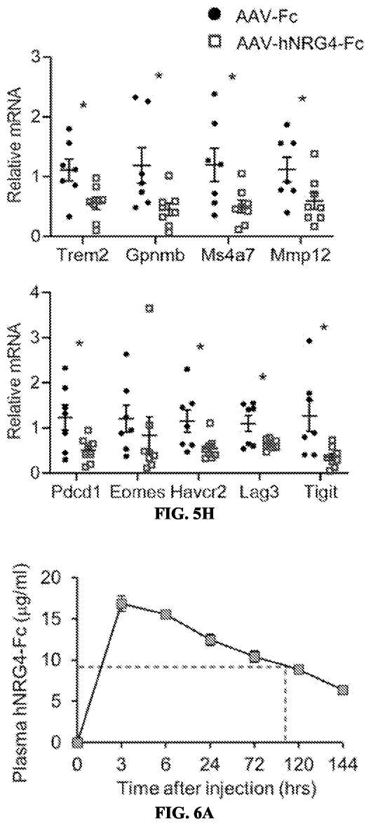

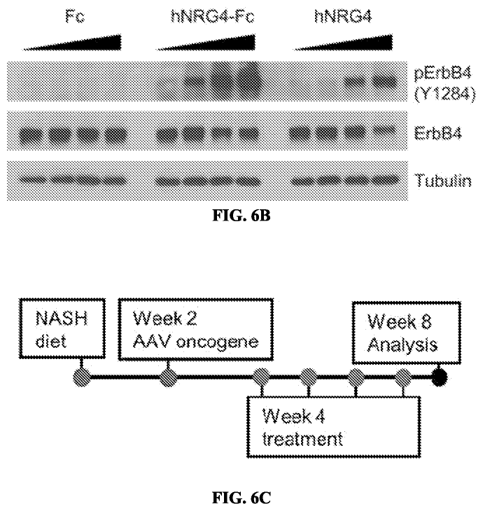

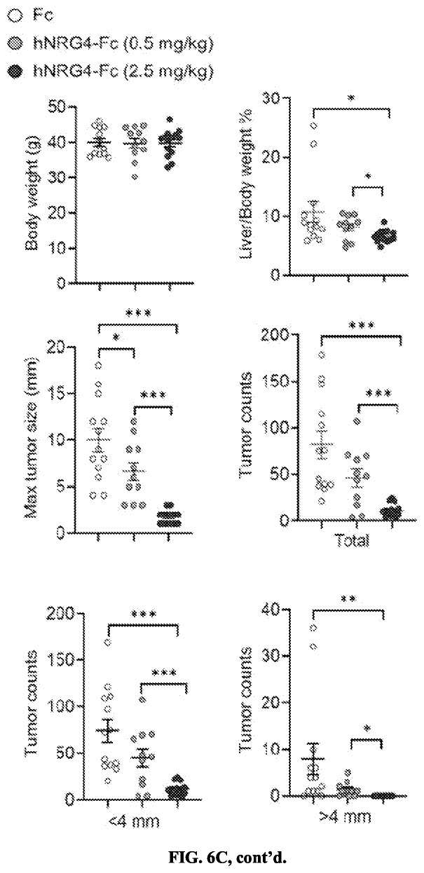

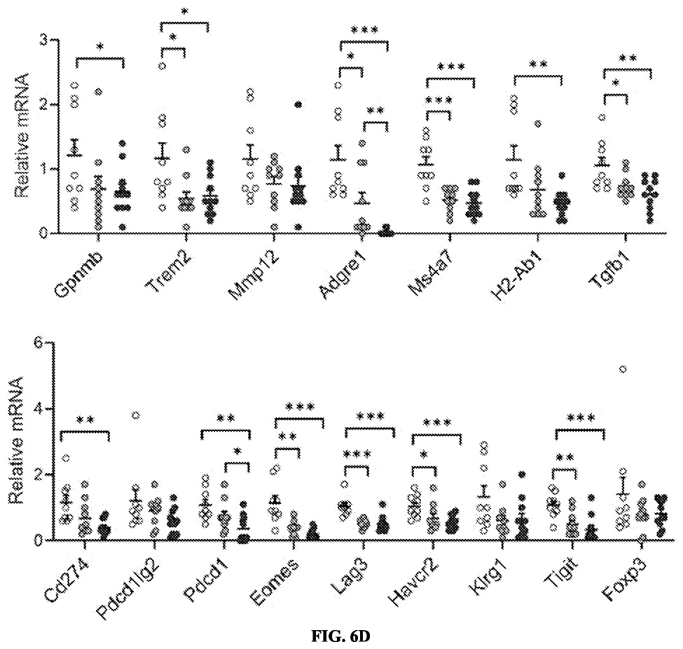

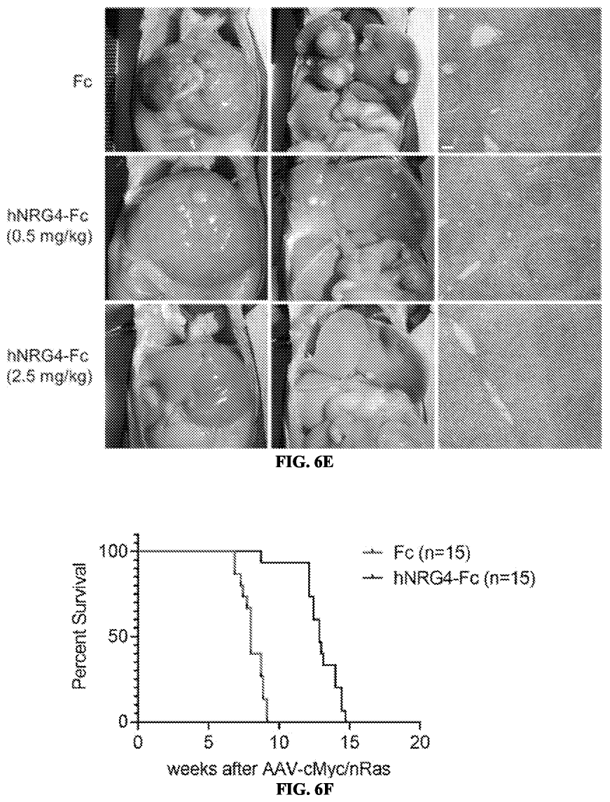

Recombinant Fc and hNRG4-Fc fusion proteins were generated and affinity purified to evaluate the therapeutic efficacy of NRG4 against NASH-HCC. hNRG4-Fc fusion protein exhibited remarkable stability in circulation with plasma half-life of approximately four days in mice (FIG. 6A). hNRG4-Fc elicits stronger ERBB4 phosphorylation compared to untagged NRG4 when tested on Min6 cells expressing the receptor, indicating that the purified fusion protein is biologically active (FIG. 6B). The efficacy of hNRG4-Fc was evaluated in liver tumorigenesis using an oncogene HCC model in combination with NASH feeding in mice. Mice on NASH diet were transduced with low doses of recombinant AAV vectors expressing cMYC and activated nRAS oncogenes. Treatments with Fc (2.5 mg/kg) or hNRG4-Fc at two doses (0.5 and 2.5 mg/kg) were initiated two weeks following oncogene transduction for four weeks before analysis (FIG. 6C). While body weight and plasma metabolite concentrations (glucose, cholesterol, triglycerides) were comparable among three groups following treatments (FIG. 13B). Hepatic gene expression analysis indicated that mRNA expression of genes associated with intrahepatic NAM induction and T cell exhaustion was significantly reduced by hNRG4-Fc (FIG. 6D).

Numerous tumor nodules were observed in Fc-treated mice (FIG. 6E). While low-dose hNRG4-Fc treatment showed a trend to have lower tumor count in treated mice, high-dose hNRG4-Fc elicited a striking reduction of the overall tumor load and maximal tumor size. Total tumor count was decreased by 86.1% in response to 2.5 mg/kg hNRG4-Fc, with small (<4 mm) tumors showing a reduction of 84.6%. Further, tumors larger than 4 mm were completely absent in mice treated with 2.5 mg/kg hNRG4-Fc. Histological analysis indicated that high-dose hNRG4-Fc treatment resulted in a notable improvement in liver appearance and histology (FIG. 6E). Suppression of liver tumorigenesis by hNRG4-Fc resulted in an approximately 60.7% increase in median survival in a separate cohort of mice (FIG. 6F). These results illustrated a potent suppressive effect of hNRG4-Fc fusion protein on HCC development in the context of NASH.

### Example 8

### Role of Liver Immune Microenvironment in Mediating the Therapeutic Efficacy of NRG4-Fc in NASH/HCC

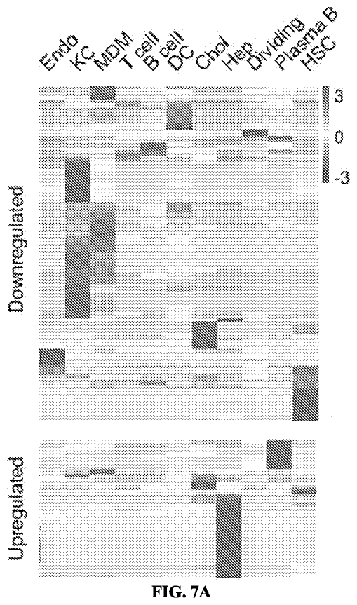

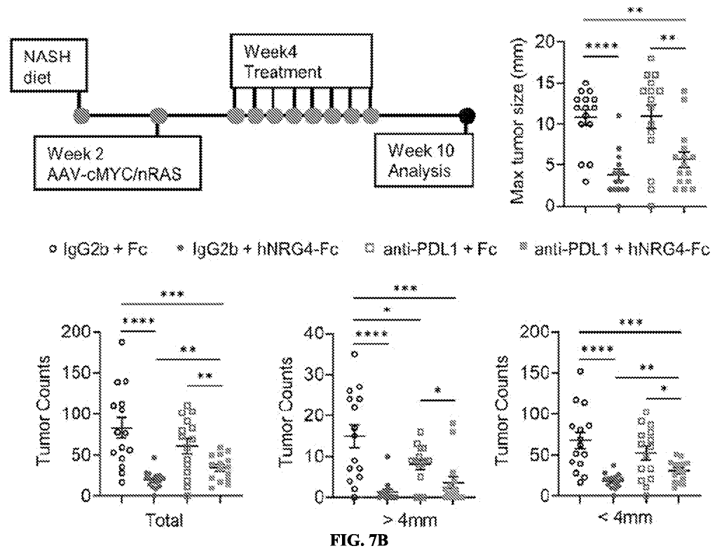

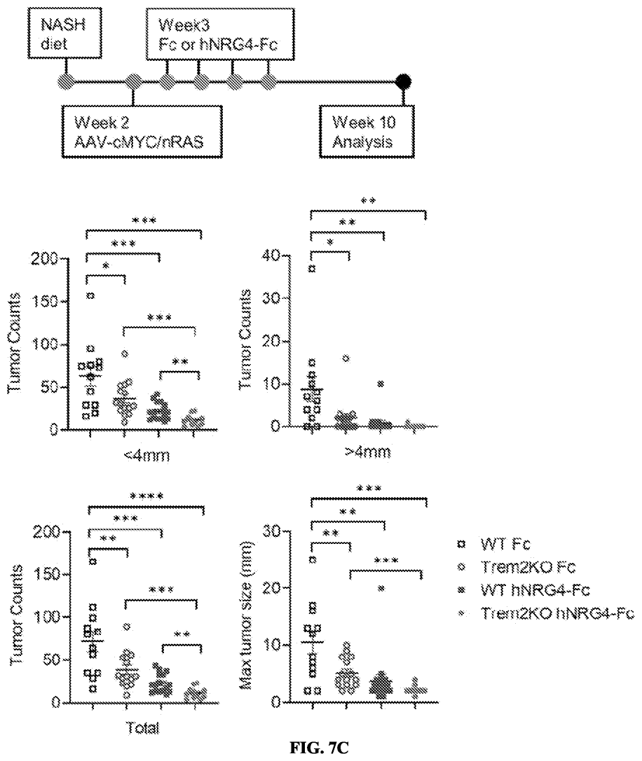

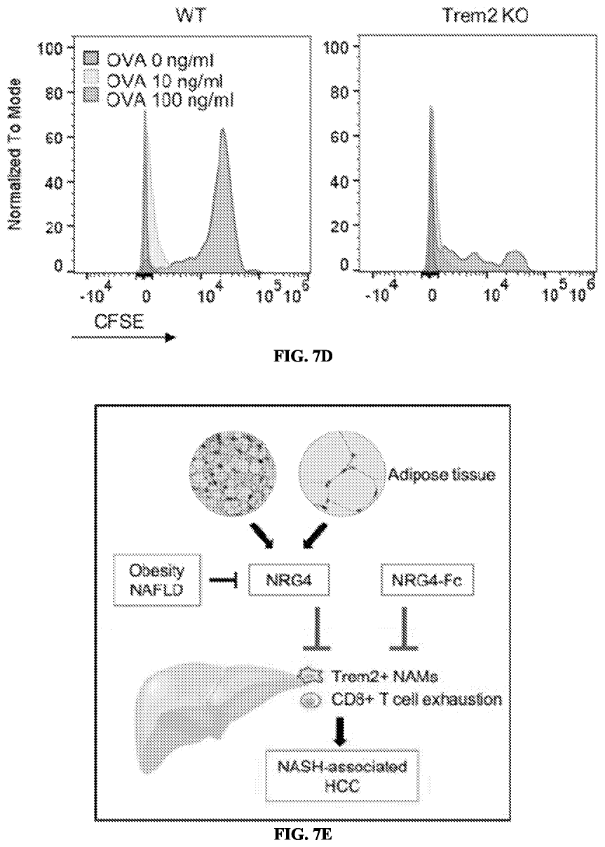

RNAseq analysis of total liver RNA isolated from mice treated with Fc or hNRG4-Fc revealed a cluster of differentially regulated genes in response to hNRG4-Fc treatment. Pathway analysis indicated that genes downregulated by hNRG4-Fc were enriched for innate immune response, cytokine signaling, and cell adhesion, while upregulated genes were enriched for carboxylic acid, lipid, and steroid metabolism. mRNA expression of NAM and T cell exhaustion markers were reduced by hNRG4-Fc. To deconvolute cell-type transcriptomic changes, a total of 1,264 genes that represent cell type markers were identified. Integration of this gene set with the bulk RNAseq data revealed that many genes downregulated by hNRG4-Fc exhibited enriched expression in KC, MDM, and HSC, while hepatocyte genes were enriched in the upregulated gene list (FIG. 7A).

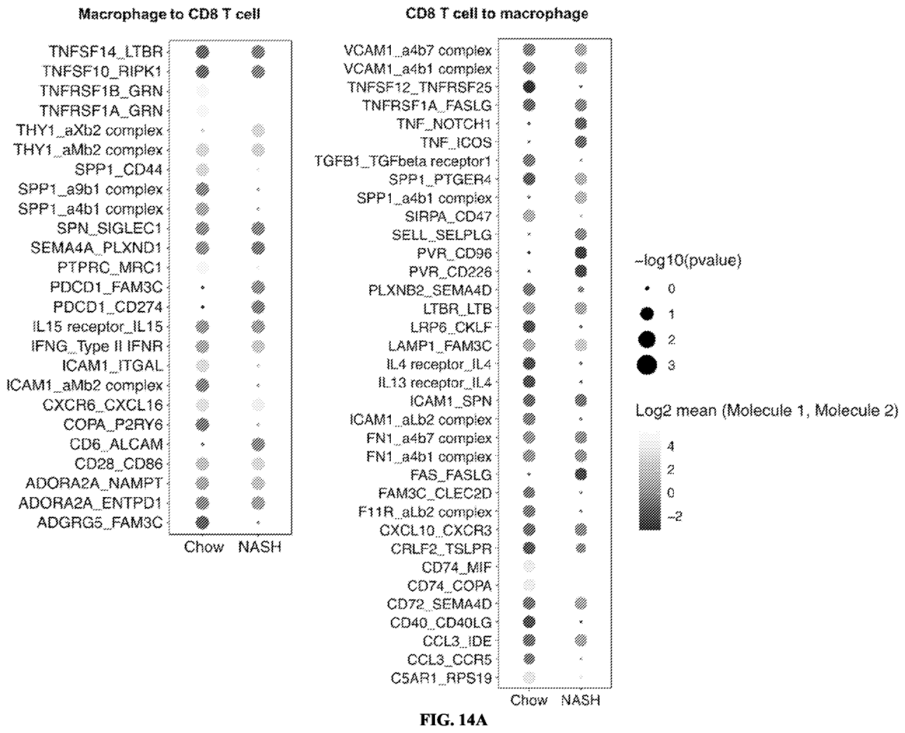

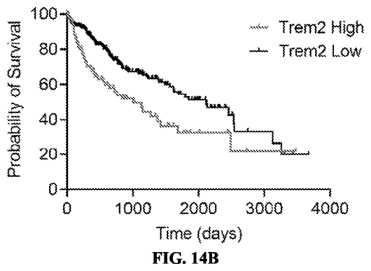

To assess whether hNRG4-Fc suppresses NASH/HCC in a cooperative manner with checkpoint inhibitors, treatment studies were performed with hNRG4-Fc and anti-PDL1 alone or in combination. Compared to control, NRG4-Fc elicited a strong tumor-suppressive effects and greatly reduced tumor counts and maximal tumor size in treated mice (FIG. 7B). No significant anti-tumor effects were observed by anti-PDL1 treatment alone. Anti-PDL1 slightly diminished the suppressive effects of hNRG4-Fc on HCC development. Recent studies have demonstrated that Trem2 is an important regulator of tumor-associated macrophages and contributes to the immunosuppressive tumor microenvironment. Trem2 inactivation restrains tumorigenesis in mice, in part through augmenting responsiveness to immunotherapy. Analysis of The Cancer Genome Atlas (TCGA) liver cancer dataset indicates that hepatic TREM2 expression is a prognostic marker for poor survival (FIG. 14B). WT and Trem2 KO mice were transduced with AAV-cMYC plus AAV-cRAS followed by four weekly treatments with Fc or hNRG4-Fc (FIG. 7C). Compared to control, liver tumor burden was significantly reduced in Trem2 KO mice and in WT mice treated with hNRG4-Fc. Remarkably, hNRG4-Fc further augmented the tumor-suppressive effects of Trem2 ablation, identifying the ability to enhance therapeutic efficacy by simultaneously targeting these two pathways.

To explore potential crosstalk between macrophages and CD8+ T cells, ligand receptor pairing analysis was performed using CellPhoneDB, a tool developed for prediction of intercellular signaling using sc-RNAseq data. The analysis revealed a network of reciprocal ligand and receptor signaling between these two cell types (FIG. 14A). Importantly, the landscape of predicted ligand and receptor interaction was profoundly altered upon NASH induction. Notably, inhibitory receptor signaling mediated by PD1 appeared to be augmented in CD8+ T cells from NASH livers. To directly test whether Trem2 plays a role in the regulation of CD8+ T cells, splenic CD8+ T cells isolated from OT-1 transgenic mice were cultured with WT or Trem2 KO BMDMs chased with ovalbumin (OVA) peptide. Interestingly, Trem2 deficient macrophages markedly enhanced CD8+ T cell proliferation in an OVA-independent manner (FIG. 7D). At low OVA concentration, a more robust proliferative response was also observed when T cells were exposed to Trem2 KO BMDMs. Together, these findings suggested that Trem2 exerts an inhibitory effect on T cell proliferation during NASH-HCC development.

### Example 9

Tumor-Suppressing Effects of hNRG4-Fc Fusion Protein in a Spontaneous Liver Cancer Model

The spontaneous liver cancer model involved administrating of a single dose of diethylnitrosamine (DEN), a chemical carcinogen, to two-week old pups, followed by NASH diet feeding for six months, beginning at two months of age. This protocol typically leads to the significant development of liver cancers in mice. After five months of NASH diet feeding, the mice were subjected to four weekly treatments of either Fc or hNRG4-Fc (1.5 mg/kg, i.p.). Analyses were performed one week after the final dose to assess the outcomes.

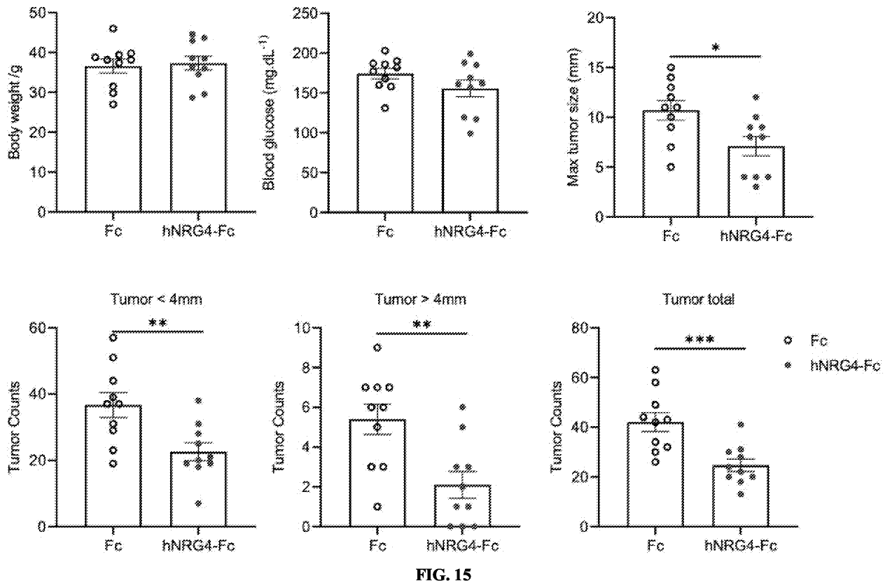

As depicted in FIG. 15, the body weight and blood glucose levels in mice treated with either Fc or hNRG4-Fc were comparable. However, administration of hNRG4-Fc resulted in a notable reduction of total tumor count and maximal tumor size compared to the Fc control group. hNRG4-Fc treatment decreased the total number of both large tumors (>4 mm) and small tumors (<4 mm). These results provide additional evidence of the therapeutic benefits of NRG4 in tumor suppression.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of treating or preventing cancer in a subject comprising administering to the subject an effective amount of a Neuregulin 4 (NRG4) polypeptide, or a nucleic acid encoding thereof.

2. The method of claim 1, wherein the NRG4 polypeptide comprises the amino acid sequence of SEQ ID NO: 1, an NRG4 variant comprising at least 70% identity to SEQ ID NO: 1, or a biologically active fragment thereof.

3. The method of claim 1 or 2, wherein the NRG4 polypeptide comprises a biologically active fragment comprising amino acids 5-46, 5-55, 5-62, 1-46, 1-55, 1-52, 1-53, 4-52, 4-53, or 1-62 of SEQ ID NO: 1.

4. The method of any of claims 1-3, wherein the NRG4 polypeptide is linked to an Fc domain.

5. The method of claim 4, wherein the Fc domain is derived from an immunoglobulin IgG Fc domain.

6. The method of claim 4 or 5, wherein the NRG4 polypeptide is linked to the Fc domain by a flexible linker.

7. The method of any of claims 1-6, wherein the NRG4 polypeptide further comprises a lipid moiety.

8. The method of any of claims 1-7, further comprising administering at least one additional therapeutic agent.

9. The method of any of claims 1-8, wherein the at least one additional therapeutic agent is an immune checkpoint inhibitor, a receptor tyrosine kinase inhibitor, or a combination thereof.

10. The method of any of claims 1-9, wherein the cancer comprises a solid tumor or hematological cancer.

11. The method of any of claims 1-10, wherein the cancer is metastatic cancer.

12. The method of any of claims 1-11, wherein the method suppresses or eliminates cancer metastasis, decreases tumor growth, prevents tumor recurrences, or any combination thereof.

13. The method of any of claims 1-12, wherein the cancer is liver cancer.

14. The method of claim 13, wherein the liver cancer is selected from: a hepatocellular carcinoma (HCC), a hepatoblastoma, a cholangiocarcinoma, a cholangiocellular cystadenocarcinoma, an angiosarcoma, a hemangioendothelioma, or a combination thereof.

15. The method of claim 13 or 14, wherein the liver cancer is hepatocellular carcinoma (HCC).

16. The method of any of claims 13-15, wherein the liver cancer is nonalcoholic steatohepatitis (NASH)-associated liver cancer.

17. The method of any of claims 1-16, wherein the subject is human.

18. The method of claim 17, wherein the subject has or is suspected of having non-alcoholic steatohepatitis.

19. A composition comprising:
a Neuregulin 4 (NRG4) polypeptide, or a nucleic acid encoding thereof; and
one or more additional therapeutic agents selected from the group consisting of: checkpoint inhibitors, receptor tyrosine kinase inhibitors, and combinations thereof.

20. The composition of claim 19, wherein the NRG4 polypeptide is linked to an Fc domain.

21. The composition of claim 20, wherein the Fc domain is derived from an immunoglobulin IgG Fc domain.

22. The method of claim 20 or 21, wherein the NRG4 polypeptide is linked to the Fc domain by a flexible linker.

23. The composition of any of claims 19-22, wherein the NRG4 polypeptide comprises the amino acid sequence of SEQ ID NO: 1, an NRG4 variant comprising at least 70% identity to SEQ ID NO: 1, or a biologically active fragment thereof.

24. The composition of any of claims 19-23, wherein the NRG4 polypeptide comprises a biologically active fragment comprising amino acids 5-46, 5-55, 5-62, 1-46, 1-55, 1-52, 1-53, 4-52, 4-53, or 1-62 of SEQ ID NO: 1.

25. The composition of any of claims 19-24, wherein the NRG4 polypeptide further comprises a lipid moiety.

26. A method of treating or preventing cancer in a subject comprising administering to the subject a composition of any of claims 19-25.

27. The method of claim 26, wherein the cancer comprises a solid tumor or hematological cancer.

28. The method of claim 26 or 27, wherein the cancer is metastatic cancer.

29. The method of any of claims 26-28, wherein the method suppresses or eliminates cancer metastasis, decreases tumor growth, prevents tumor recurrences, or any combination thereof.

30. The method of any of claims 26-29, wherein the cancer is liver cancer.

31. The method of claim 30, wherein the liver cancer is selected from: a hepatocellular carcinoma (HCC), a hepatoblastoma, a cholangiocarcinoma, a cholangiocellular cystadenocarcinoma, an angiosarcoma, a hemangioendothelioma, or a combination thereof.

32. The method of claim 30 or 31, wherein the liver cancer is hepatocellular carcinoma (HCC).

33. The method of any of claims 30-32, wherein the liver cancer is NASH-associated liver cancer.

34. The method of any of claims 26-33, wherein the subject is human.

35. Use of a composition of any of claims 19-25 for the treatment or prevention of cancer.

36. Use of a Neuregulin 4 (NRG4) polypeptide for the treatment or prevention of cancer

37. The use of claim 35 or 36, wherein the cancer is liver cancer.

38. The use of claim 37, wherein the liver cancer is selected from: a hepatocellular carcinoma (HCC), a hepatoblastoma, a cholangiocarcinoma, a cholangiocellular cystadenocarcinoma, an angiosarcoma, a hemangioendothelioma, or a combination thereof.

39. The use of claim 37 or 38, wherein the liver cancer is hepatocellular carcinoma (HCC).

40. The use of any of claims 37-39, wherein the liver cancer is NASH-associated liver cancer.

41. The use of any of claims 35-40, wherein the NRG4 polypeptide is linked to an Fc domain.


SEQUENCE LISTING (3 sequences)

SEQ ID NO: 1 - protein, 115 aa, Homo sapiens
MPTDHEEPCGPSHKSFCLNGGLCYVIPTIPSPFCRCVENYTGARCEEVFLPGSSIQTKSNLFEAFVALAVLVTLIIGAFYFLCRKGHFQRASSVQYDINLVETSSTSAHHSHEQH

SEQ ID NO: 2 - protein, 115 aa, Mus musculus
MPTDHEQPCGPRHRSFCLNGGICYVIPTIPSPFCRCIENYTGARCEEVFLPSSSIPSESNLSAAFVVLAVLLTLTIAALCFLCRKGHLQRASSVQCEISLVETNNTRTRHSHREH

SEQ ID NO: 3 - protein, 6 aa, synthetic construct
GGGGGS